(19) United States
(12) Patent Application Publication
(10) Pub. No.: US 2011/0015182 A1
(43) Pub. Date: Jan. 20, 2011

(54) NOVEL CARBAMATE AMINO ACID AND PEPTIDE PRODRUGS OF OPIOIDS AND USES THEREOF

(76) Inventors: Richard Franklin, Fleet (GB); Bernard T. Golding, Newcastle upon Tyne (GB); Robert G. Tyson, Durham (GB)

Correspondence Address: MCDERMOTT WILL & EMERY LLP, 600 13TH STREET, N.W., WASHINGTON, DC, 20005-3096, US

(21) Appl. No.: 12/837,788

(22) Filed: Jul. 16, 2010

Related U.S. Application Data
(60) Provisional application No. 61/271,185, filed on Jul. 17, 2009.

(51) Int. Cl. A61K 31/55; C07D 223/04; C07D 405/12; C07D 403/12; C07D 411/12; C07D 223/10; A61P 25/04

(52) U.S. Cl. 514/217.08; 540/611; 514/212.01; 540/596; 514/217.03; 540/602; 540/531

(57) ABSTRACT

Carbamate linked prodrugs of meptazinol and other opioid analgesics are provided. The prodrug moiety may comprise a single amino acid or short peptide. Additionally, the present invention relates to methods for reducing gastrointestinal side effects in a subject, the gastrointestinal side effects being associated with the administration of an opioid analgesic. The methods comprise orally administering an opioid prodrug or pharmaceutically acceptable salt thereof to a subject, wherein the opioid prodrug is comprised of an opioid analgesic covalently bonded through a carbamate linkage to a prodrug moiety, and wherein upon oral administration, the prodrug or pharmaceutically acceptable salt minimizes at least one gastrointestinal side effect associated with oral administration of the opioid analgesic alone. Compositions for use with the method are also provided.

This application claims priority to provisional application No. 61/271,185, filed Jul. 17, 2009, the contents of which are hereby incorporated by reference in their entirety.

### FIELD OF THE INVENTION

The present invention relates to the utilization of amino acid and small peptide prodrugs of meptazinol, oxymorphone, buprenorphine and other opioid analgesics, to reduce or eliminate pain, to increase the oral availability of the respective opioid analgesic, and/or to reduce the opioid analgesic's adverse gastrointestinal (GI) side effects, including constipation and vomiting.

### BACKGROUND OF THE INVENTION

Appropriate treatment of pain continues to represent a major problem for both subjects and healthcare professionals. Optimal pharmacologic management of pain requires selection of the appropriate analgesic drug that achieves rapid efficacy with minimal side effects.

Analgesics for treating mild pain are readily available, both over the counter (OTC) and by prescription. These include aspirin, ibuprofen and acetaminophen (paracetamol). While these agents are well established for the treatment of mild pain, they are not without their side effects. For example, aspirin may cause local stomach irritation and paracetamol, in excessives doses, is associated with marked liver toxicity followed potentially by liver failure.

More effective analgesics such as the stronger non-steroidal anti inflammatory drugs, (e.g., ketoprofen, diclofenac and naproxen), while offering effective pain relief in moderate pain, may have more pronounced side effects such as gastric ulceration and possible hemorrhage.

Treatment of more severe pain with opioid analgesics such as oxyocodone, oxymorphone, hydromorphone and morphine offers good analgesia, but each is beset by problems of gastrointesinal (GI) tract intolerance and adverse reactions. These adverse GI reactions include nausea, dyspepsia, vomiting, gastric ulceration, diarrhea and constipation, and, in some cases, a combination of these reactions.

Additionally, treatment of more severe pain with opioid analgesics such as oxymorphone may also have other limitations. Unwanted effects can include sedation, respiratory depression, chronic constipation and abuse liability.

Many of the stronger opioid analgesics possess a phenolic or hydroxylic function. Such drugs include butorphanol, buprenorphine, codeine, dezocine, dihydrocodeine, hydromorphone, levorphanol, meptazinol, morphine, nalbuphine, oxycodone, oxymorphone, and pentazocine. As a consequence of the presence of either a phenolic or hydroxylic function, many of these compounds are subject to extensive metabolism during the initial passage through the liver after oral dosing, limiting the amount of unchanged drug which can reach the systemic circulation. This high first pass effect results in poor oral bioavailability. For example, meptazinol, oxymorphone and buprenorphine all have oral bioavailabilities less than 10%. A direct consequence of such low bioavailability is considerable variability in attained blood levels within and between subjects. For example, with meptazinol, the range of observed oral bioavailabilities extends from 2-20% (Norbury et al., (1983) Eur. J Clin Pharmacol 25, 77-80). This inevitably results in a variable analgesic response requiring subjects to be individually titrated to achieve adequate pain relief. Dose titration can be tedious and time consuming and make effective treatment of subjects extremely difficult. In any event, the treatment of moderate to severe pain demands urgent relief and subjects may not be prepared to tolerate a protracted period of dose titration. This inevitably leads to compliance issues among subjects.

Peptide prodrugs of various opioids have been synthesized previously and are described in, for example, International Patent Application Publication Nos. WO 05/032474, WO 07/126832 and WO 02/034237, WO 03/020200, WO 03/072046, WO 07/030577 and WO 2007/120648.

The current oral formulations of meptazinol, oxymorphone as well as the currently available formulations of buprenorphine are not ideal for pain relief. Thus, there is clearly an important need for improved oral formulations of these and other hydroxylic analgesics, in order to increase the respective analgesic's oral bioavailability, as well as to deliver a pharmacologically effective amount of the drug for the treatment of pain and other analgesic benefits. Additionally, there is clearly still a need for a pharmaceutical product capable of relieving severe pain but without the GI side effects which currently blight all the major strong opioid analgesics. The present invention addresses these and other needs.

### SUMMARY OF THE INVENTION

In one embodiment, the present invention is directed to an opioid prodrug of Formula I

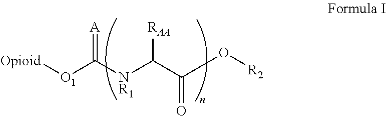

or a pharmaceutically acceptable salt thereof, wherein

O₁ is a hydroxylic oxygen (e.g., phenolic oxygen) present in the unbound opioid molecule,

A is selected from O and S,

each occurrence of R₁ is independently hydrogen, alkyl or substituted alkyl,

R₂ is selected from a C₁-C₄ alkyl, an amino acid (e.g., serine (—CH₂CH(NH₂)COOH)), a substituted phenyl group (e.g., substituted with a carboxyl group, such as 2-COOH-phenyl) and a substituted alkyl group,

n is an integer from 1 to 9 (e.g., n can be 1),

each occurrence of R_{AA} is independently a proteinogenic or non-proteinogenic amino acid side chain, and

the opioid is selected from butorphanol, buprenorphine, codeine, dezocine, dihydrocodeine, hydromorphone, levorphanol, meptazinol, morphine, nalbuphine, oxycodone, oxymorphone, and pentazocine, or active metabolites thereof (e.g., ethyl-hydroxylated meptazinol (3-[3-(2-Hydroxy-ethyl)-1-methyl-perhydro-azepin-3-yl]-phenol), ethyl-carboxylated meptazinol (3-[3-(2-carboxy-ethyl)-1-methyl-perhydro-azepin-3-yl]-phenol), des-methyl meptazinol, 2-oxomeptazinol and 7-oxomeptazinol).

In one Formula I embodiment, R₂ is selected from t-butyl, isopropyl, ethyl, methyl,

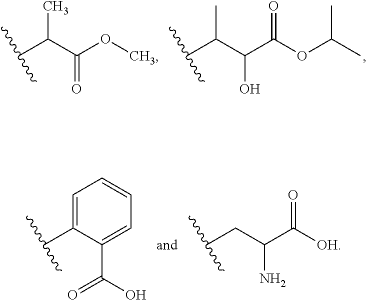

In another embodiment, R₂ is not t-butyl. In another embodiment, R₂ is methyl, ethyl, or isopropyl.

In yet another embodiment, the present invention is directed to an opioid prodrug of Formula II:

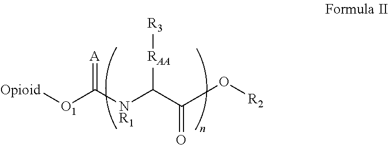

or a pharmaceutically acceptable salt thereof, wherein

O₁ is a hydroxylic oxygen present in the unbound opioid molecule,

A is selected from O and S,

R₁ is H, alkyl or substituted alkyl,

R₂ is selected from H, cycloalkyl, aryl, substituted cycloalkyl, alkyl, substituted alkyl group and an opioid,

If R₂ is an opioid, —O— is a hydroxylic oxygen present in the unbound opioid,

n is an integer from 1 to 9 (e.g., n can be 1),

R_{AA} is a proteinogenic or non-proteinogenic amino acid side chain, and each occurrence of R_{AA} can be the same or different,

each occurrence of R₃ is independently absent or an amino acid (e.g., cysteine), each amino acid R₃ is bonded to R_{AA} via a side chain, N-terminus or C-terminus of the amino acid, and

the opioid is selected from butorphanol, buprenorphine, codeine, dezocine, dihydrocodeine, hydromorphone, levorphanol, meptazinol, morphine, nalbuphine, oxycodone, oxymorphone, and pentazocine, or active metabolites thereof (e.g., ethyl-hydroxylated meptazinol (3-[3-(2-Hydroxy-ethyl)-1-methyl-perhydro-azepin-3-yl]-phenol), ethyl-carboxylated meptazinol (3-[3-(2-carboxy-ethyl)-1-methyl-perhydro-azepin-3-yl]-phenol), des-methyl meptazinol, 2-oxomeptazinol and 7-oxomeptazinol).

In one Formula II embodiment, the opioid is meptazinol, R₂ is meptazinol, R₃ is absent and n is 1. In a further embodiment, R_{AA} is a valine side chain.

In another embodiment, the present invention is directed to compounds of Formula III:

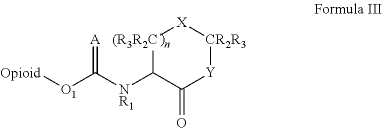

or a pharmaceutically acceptable salt thereof, wherein,

A and Y are independently selected from O and S,

X is absent or selected from O and S,

O₁ is a hydroxylic oxygen present in the unbound opioid molecule,

R₁ is H, alkyl or substituted alkyl,

R₂ and R₃ are independently selected from hydrogen, aryl, unsubstituted alkyl and substituted alkyl,

n is an integer from 1 to 4 (e.g., n can be 1), and

the opioid is selected from butorphanol, buprenorphine, codeine, dezocine, dihydrocodeine, hydromorphone, levorphanol, meptazinol, morphine, nalbuphine, oxycodone, oxymorphone, and pentazocine, or active metabolites thereof (e.g., ethyl-hydroxylated meptazinol (3-[3-(2-Hydroxy-ethyl)-1-methyl-perhydro-azepin-3-yl]-phenol), (3-[3-(2-carboxy-ethyl)-1-methyl-perhydro-azepin-3-yl]-phenol), des-methyl meptazinol, 2-oxomeptazinol and 7-oxomeptazinol).

In one embodiment, the opioid prodrugs of the present invention are directed to compounds of Formula IV:

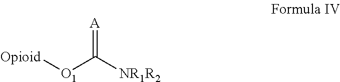

or a pharmaceutically acceptable salt thereof, wherein,

O₁ is a hydroxylic oxygen present in the unbound opioid molecule,

A is selected from O and S,

R₁ and R₂ are independently selected from hydrogen, aryl, alkyl, and substituted alkyl group, and

the opioid is selected from butorphanol, buprenorphine, codeine, dezocine, dihydrocodeine, hydromorphone, levorphanol, meptazinol, morphine, nalbuphine, oxycodone, oxymorphone, and pentazocine, or active metabolites thereof (e.g., ethyl-hydroxylated meptazinol (3-[3-(2-Hydroxy-ethyl)-1-methyl-perhydro-azepin-3-yl]-phenol), ethyl-carboxylated meptazinol (3-[3-(2-carboxy-ethyl)-1-methyl-perhydro-azepin-3-yl]-phenol), des-methyl meptazinol, 2-oxomeptazinol and 7-oxomeptazinol).

In one embodiment, R₁ and R₂ are independently hydrogen or C₁-C₄ alkyl, optionally substituted by —COOH, halogen, amino, mono-(C₁-C₄ alkyl)amino, di-(C₁-C₄ alkyl)amino, —NHC(O)—C₁-C₄ alkyl, phenyl, or C₁-C₄ alkoxy. According to another embodiment, R₁ is hydrogen and R₂ is C₁-C₄ alkyl. According to another embodiment, R₁ and R₂ are independently C₁-C₄ alkyl.

In one embodiment, the opioid prodrugs of the present invention are directed to compounds of Formula V:

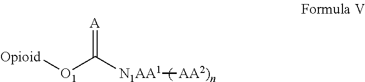

or a pharmaceutically acceptable salt thereof, wherein,

O₁ is a hydroxylic oxygen present in the unbound opioid molecule,

A is selected from O and S,

AA¹ is selected from a proteinogenic amino acid, a β-amino acid (e.g., β-alanine) and pyroglutamic acid,

AA² is an α- or β-amino acid (e.g., valine),

n is an integer from 0 to 9;

N₁ is a nitrogen atom present in the first AA, and

the opioid is selected from butorphanol, buprenorphine, codeine, dezocine, dihydrocodeine, hydromorphone, levorphanol, meptazinol, morphine, nalbuphine, oxycodone, oxymorphone, and pentazocine, or active metabolites thereof (e.g., ethyl-hydroxylated meptazinol (3-[3-(2-Hydroxy-ethyl)-1-methyl-perhydro-azepin-3-yl]-phenol), ethyl-carboxylated meptazinol (3-[3-(2-carboxy-ethyl)-1-methyl-perhydro-azepin-3-yl]-phenol), des-methyl meptazinol, 2-oxomeptazinol and 7-oxomeptazinol).

In one Formula V embodiment, N₁ is the nitrogen atom of β-alanine.

In one Formula V embodiment, N₁ is the nitrogen atom of pyroglutamate and n is 0.

In one embodiment, the opioid prodrugs of the present invention are directed to compounds of Formula Va:

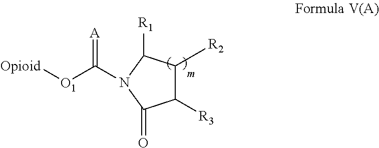

or a pharmaceutically acceptable salt thereof, wherein,

O₁ is a hydroxylic oxygen present in the unbound opioid molecule,

A is selected from O and S,

R₁, R₂ and R₃ are independently selected from hydrogen, aryl, alkyl, substituted alkyl group and carboxyl, and at least one occurrence of R₁, R₂ and R₃ is carboxyl,

m is an integer from 1 to 3; and

the opioid is selected from butorphanol, buprenorphine, codeine, dezocine, dihydrocodeine, hydromorphone, levorphanol, meptazinol, morphine, nalbuphine, oxycodone, oxymorphone, and pentazocine, or active metabolites thereof (e.g., ethyl-hydroxylated meptazinol (3-[3-(2-Hydroxy-ethyl)-1-methyl-perhydro-azepin-3-yl]-phenol), ethyl-carboxylated meptazinol (3-[3-(2-carboxy-ethyl)-1-methyl-perhydro-azepin-3-yl]-phenol), des-methyl meptazinol, 2-oxomeptazinol and 7-oxomeptazinol).

In one Formula V(A) embodiment, at least one carboxyl moiety of R₁, R₂ or R₃ is bound to an amino acid or peotide.

In yet another embodiment, the present invention is directed to an opioid prodrug of Formula VI:

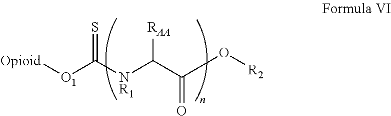

or a pharmaceutically acceptable salt thereof, wherein,

O₁ is a hydroxylic oxygen present in the unbound opioid molecule,

R₁ and R₂ are independently selected from hydrogen, unsubstituted alkyl, substituted alkyl, cycloalkyl, or substituted cycloalkyl group,

n is an integer from 1 to 9,

each occurrence of R_{AA} is independently a proteinogenic or non-proteinogenic amino acid side chain (e.g., R_{AA} can be isopropyl), and

the opioid is selected from butorphanol, buprenorphine, codeine, dezocine, dihydrocodeine, hydromorphone, levorphanol, meptazinol, morphine, nalbuphine, oxycodone, oxymorphone, and pentazocine, or active metabolites thereof (e.g., ethyl-hydroxylated meptazinol (3-[3-(2-Hydroxy-ethyl)-1-methyl-perhydro-azepin-3-yl]-phenol), (3-[3-(2-carboxy-ethyl)-1-methyl-perhydro-azepin-3-yl]-phenol), des-methyl meptazinol, 2-oxomeptazinol and 7-oxomeptazinol).

R₂ in one embodiment is hydrogen or C₁-C₄ alkyl.

In one embodiment, R_{AA} is isopropyl and the carbon atom attached to R_{AA} is in the S configuration.

In yet another embodiment, the present invention is directed to an opioid prodrug of Formula VII:

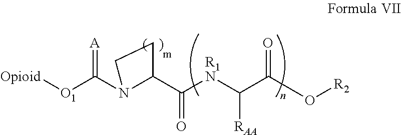

or a pharmaceutically acceptable salt thereof, wherein

O₁ is a hydroxylic oxygen present in the unbound opioid molecule,

A is selected from O and S,

each occurrence of R₁ is independently hydrogen, alkyl or substituted alkyl,

m is an integer from 1 to 4 and n is an integer from 0 to 9,

R₂ is selected from hydrogen, C₁-C₄ alkyl, an amino acid (e.g., serine (—CH₂CH(NH₂)COOH)), or a substituted phenyl group (e.g., substituted with a carboxyl group, such as 2-COOH-phenyl) and an opioid,

If R₂ is an opioid, —O— is a hydroxylic oxygen present in the unbound opioid,

each occurrence of R_{AA} is independently a proteinogenic or non-proteinogenic amino acid side chain, and

the opioid is selected from butorphanol, buprenorphine, codeine, dezocine, dihydrocodeine, hydromorphone, levorphanol, meptazinol, morphine, nalbuphine, oxycodone, oxymorphone, and pentazocine, or active metabolites thereof (e.g., ethyl-hydroxylated meptazinol (3-[3-(2-Hydroxy-ethyl)-1-methyl-perhydro-azepin-3-yl]-phenol), (3-[3-(2-carboxy-ethyl)-1-methyl-perhydro-azepin-3-yl]-phenol), des-methyl meptazinol, 2-oxomeptazinol and 7-oxomeptazinol).

In one Formula VII embodiment, R₂ is not hydrogen.

In one Formula VII embodiment, R₁ is hydrogen, m is 2, n is 1 and R₂ is hydrogen. In this embodiment, the prodrug moiety is proline carbamate.

In yet another embodiment, the present invention is directed to an opioid prodrug of Formula VIII:

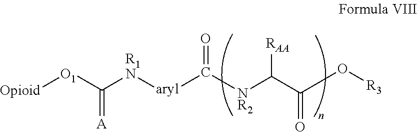

or a pharmaceutically acceptable salt thereof,

O₁ is a hydroxylic oxygen present in the unbound opioid molecule,

R₁ is selected from hydrogen, alkyl, substituted alkyl, cycloalkyl and substituted cycloalkyl group,

Each occurrence of R₂ is independently selected from hydrogen, alkyl, substituted alkyl, cycloalkyl, and substituted cycloalkyl group,

R₃ is selected from hydrogen, alkyl, substituted alkyl, cycloalkyl, substituted cycloalkyl group and an opioid,

If R₃ is an opioid, —O— is a hydroxylic oxygen present in the unbound opioid,

NR₁ and the carboxyl group immediately flanking the aryl group in Formula VIII can be a part of the aryl group,

n is an integer from 1 to 9,

each occurrence of R_{AA} is independently a proteinogenic or non-proteinogenic amino acid side chain (e.g., R_{AA} can be isopropyl) and

the opioid is selected from butorphanol, buprenorphine, codeine, dezocine, dihydrocodeine, hydromorphone, levorphanol, meptazinol, morphine, nalbuphine, oxycodone, oxymorphone, and pentazocine, or active metabolites thereof (e.g., ethyl-hydroxylated meptazinol (3-[3-(2-Hydroxy-ethyl)-1-methyl-perhydro-azepin-3-yl]-phenol), ethyl-carboxylated meptazinol (3-[3-(2-carboxy-ethyl)-1-methyl-perhydro-azepin-3-yl]-phenol), des-methyl meptazinol, 2-oxomeptazinol and 7-oxomeptazinol).

In a further Formula VIII embodiment, the moiety is selected

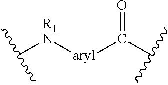

from

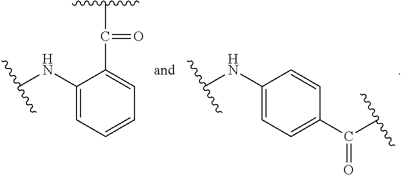

Yet another embodiment is an opioid prodrug selected from those listed below and pharmaceutically acceptable salts thereof. It is to be understood that these compounds use meptazinol for illustrative purposes, and that one of ordinary skill in the art can readily substitute other opioids with a hydroxylic function, for meptazinol. It is also with the ordinary skill in the art to change the amino acid moiety, e.g., from valine to another proteinogenic or non-proteinogenic amino acid or peptide.

| | Prodrug | Structure |
|---|---|---|
| 1 | MVC tert-Butyl ester | 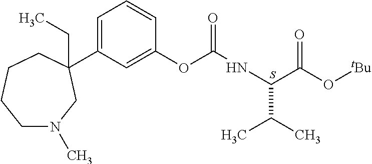 |
| 2 | MVC Isopropyl ester | 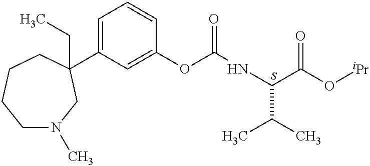 |
| 3 | MVC ethyl ester | 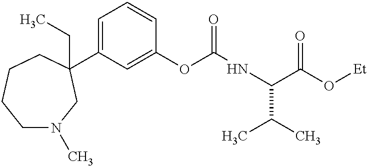 |
| 4 | MVC [isopropyl(S)-lactate] ester | 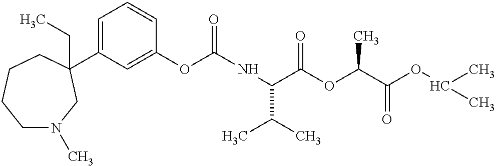 |
| 5 | MVC Salicyclic acid ester | 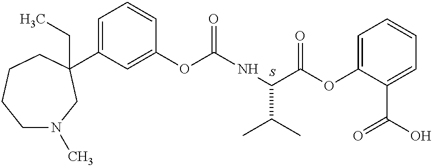 |
| 6 | MVC (S)-serine ester | 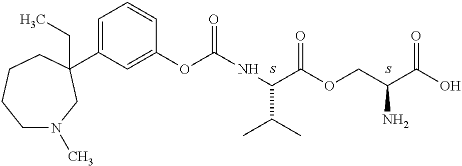 |
| 7 | Meptazinol homo-serine lactone carbamate | 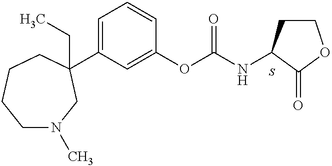 |
| 8 | Meptazinol aminomalonic acid carbamate | 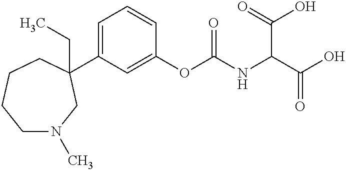 |
| 9 | Meptazinol cystine carbamate | 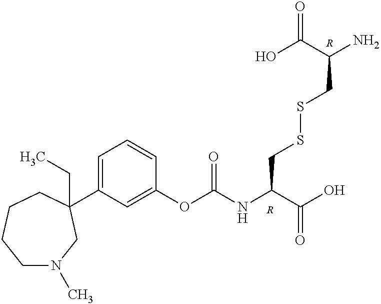 |
| 10 | Meptazinol β-alanine-valine carbamate | 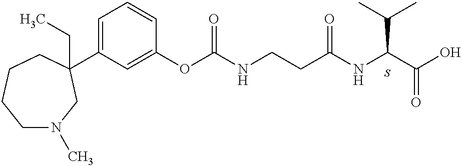 |
| 11 | Meptazinol mono-propyl carbamate | 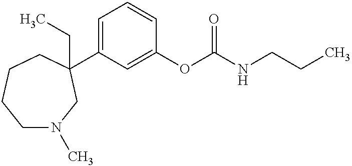 |
| 12 | Meptazinol di-propyl carbamate | 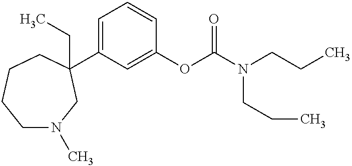 |
| 13 | Meptazinol sarcosine carbamate | 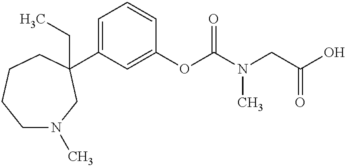 |
| 14 | Meptazinol (O-methyl serine) carbamate | 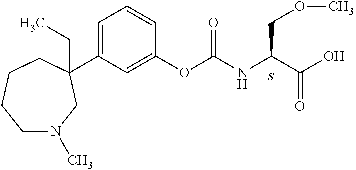 |
| 15 | Meptazinol β-(acetylamino)alanine carbamate | 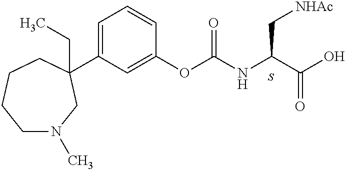 |
| 16 | Meptazinol β-aminoalanine carbamate | 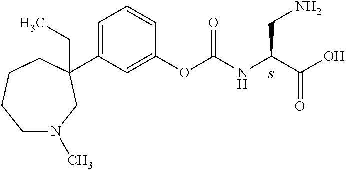 |
| 17 | Meptazinol (isopropylidene-threonine) carbamate | 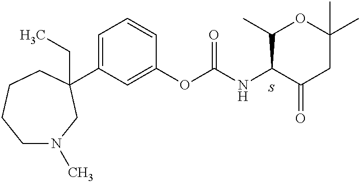 |
| 18 | Meptazinol phenylglycine carbamate | 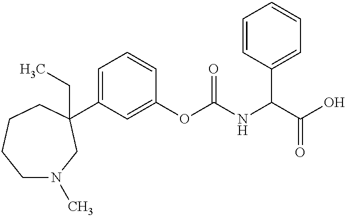 |
| 19 | Meptazinol proline carbamate | 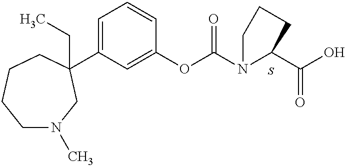 |
| 20 | Meptazinol (issopropylidene0cysteine) carbamate | 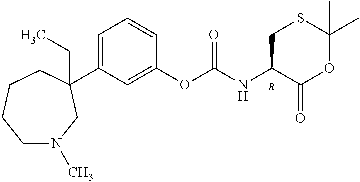 |
| 21 | Meptazinol (isopropylidene-homo-cysteine) carbamate | 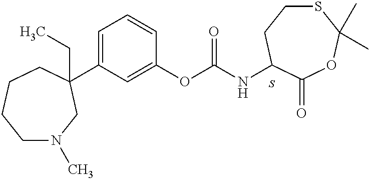 |
| 22 | Meptazinol β-chloroalanine carbamate | 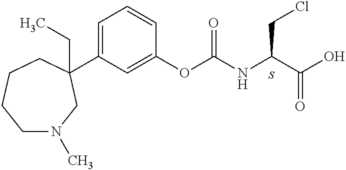 |
| 23 | Des-methyl meptazinol-S-valine carbamate | 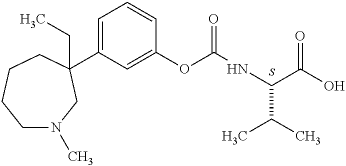 |
| 24 | 2-Oxomeptazinol-S-valine carbamate | 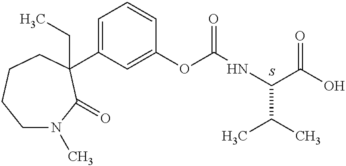 |
| 25 | 7-Oxomeptazinol-S-valine carbamate | 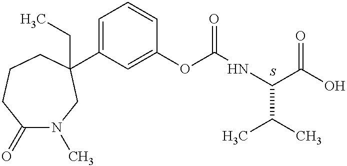 |
| 26 | Meptazinol valine thiocarbamate | 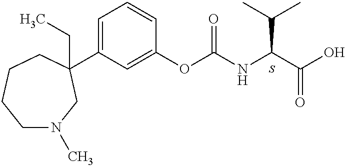 |
| 27 | Meptazinol valine-lysine side-chain carbamate H-Val-Lys(CO.OMeptazinol)-OH | 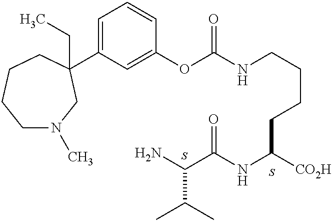 |
| 28 | Meptazinol pyroglutamate carbamate | 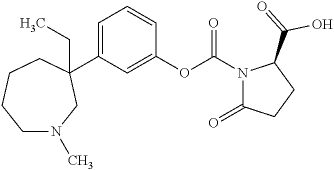 |
| 29 | Bis-Meptazinol valine carbamate | 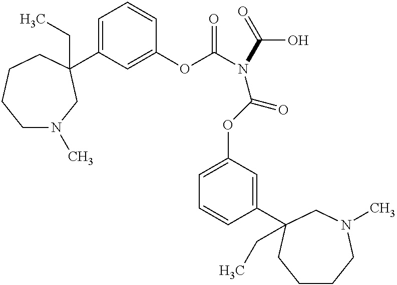 |
| 30 | Meptazinol para aminobenzoic acid valine carbamate | 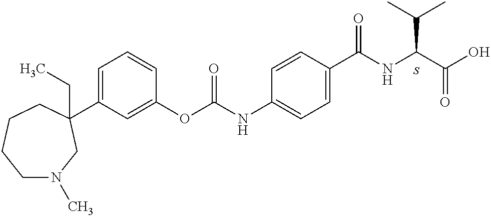 |

In yet another embodiment, the present invention is directed to a pharmaceutical composition comprising one or more of the opioid prodrugs of the present invention, and one or more pharmaceutically acceptable excipients.

Yet another embodiment is a method of reducing or eliminating pain by administering, to a subject in need thereof, an effective amount of the opioid prodrug of the present invention, or a pharmaceutical composition of the present invention.

In a further embodiment, the type of pain which can be treated with the opioid prodrugs of the present invention includes neuropathic pain and nociceptive pain. Other specific types of pain which can be treated with the opioid prodrugs of the present invention include, but are not limited to, acute pain, chronic pain, post-operative pain, pain due to neuralgia (e.g., post herpetic neuralgia or trigeminal neuralgia), pain due to diabetic neuropathy, dental pain, pain associated with arthritis or osteoarthritis, and pain associated with cancer or its treatment.

Another embodiment is a method of treating a disorder in a subject in need thereof with an opioid without inducing gastrointestinal side effects associated with the opioid. The method comprises orally administering an effective amount of an opioid prodrug of the present invention to the subject. The disorder may be one treatable with an opioid. For example, the disorder may be pain, such as neuropathic pain or nociceptive pain. Other specific types of pain which can be treated with the opioid prodrugs of the present invention include, but are not limited to, acute pain, chronic pain, post-operative pain, pain due to neuralgia (e.g., post herpetic neuralgia or trigeminal neuralgia), pain due to diabetic neuropathy, dental pain, pain associated with arthritis or osteoarthritis, and pain associated with cancer or its treatment.

In a further embodiment, the GI side effect associated with administration of an opioid analgesic is selected from, but is not limited to nausea, dyspepsia, post operative ileus, vomiting, constipation, or a combination of these side effects.

### DETAILED DESCRIPTION OF THE INVENTION

### Definitions

As used herein:

The term “peptide” refers to an amino acid chain consisting of 2 to 9 amino acids, unless otherwise specified. In preferred embodiments, the peptide used in the present invention is 2 or 3 amino acids in length.

The term “amino acid” refers both to proteinogenic and non-proteinogenic amino acids, and carbamate derivatives thereof.

A “proteinogenic amino acid” is one of the twenty two amino acids used for protein biosynthesis as well as other amino acids which can be incorporated into proteins during translation. A proteinogenic amino acid generally has the formula

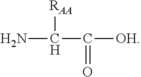

R_{AA} is referred to as the amino acid side chain, or in the case of a proteinogenic amino acid, as the proteinogenic amino acid side chain. The proteinogenic amino acids include glycine, alanine, valine, leucine, isoleucine, aspartic acid, glutamic acid, serine, threonine, glutamine, asparagine, arginine, lysine, proline, phenylalanine, tyrosine, tryptophan, cysteine, methionine, histidine, selenocysteine and pyrrolysine.

Examples of proteinogenic amino acid sidechains include hydrogen (glycine), methyl (alanine), isopropyl (valine), sec-butyl (isoleucine), —CH₂CH(CH₃)₂ (leucine), benzyl (phenylalanine), p-hydroxybenzyl (tyrosine), —CH₂OH (serine), —CH(OH)CH₃ (threonine), —CH₂-3-indoyl (tryptophan), —CH₂COOH (aspartic acid), —CH₂CH₂COOH (glutamic acid), —CH₂C(O)NH₂ (asparagine), —CH₂CH₂C(O)NH₂ (glutamine), —CH₂SH, (cysteine), —CH₂CH₂SCH₃ (methionine), —(CH₂)₄NH₂ (lysine), —(CH₂)₃NHC(═NH)NH₂ (arginine) and —CH₂-3-imidazoyl (histidine).

A “non-proteinogenic amino acid” is an organic compound that is not among those encoded by the standard genetic code, or incorporated into proteins during translation. Non-proteinogenic amino acids, thus, include amino acids or analogs of amino acids other than the 20 proteinogenic amino acids and include all possible stereoisomers, and mixtures thereof (e.g., racemeic mixtures). Non-proteinogenic amino acids also includes d-isomers of proteinogenic amino acids. Additionally, amino acids are included in the definition on “non-proteinogenic amino acids.”

Examples of non-proteinogenic amino acids include, but are not limited to: citrulline, homocitrulline, hydroxyproline, homoarginine, homoproline, ornithine, 4-amino-phenylalanine, norleucine, cyclohexylalanine, α-aminoisobutyric acid, acetic acid, O-methyl serine (i.e., an amino acid sidechain having the formula

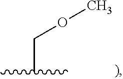

N-methyl-alanine, N-methyl-glycine, N-methyl-glutamic acid, tert-butylglycine, α-aminobutyric acid, tert-butylalanine, α-aminoisobutyric acid, 2-aminoisobutyric acid 2-aminoindane-2-carboxylic acid, selenomethionine, acetylamino alanine (i.e., an amino acid sidechain having the formula

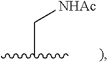

β-alanine, β-(acetylamino)alanine, β-aminoalanine, β-chloroalanine, phenylglycine, lanthionine, dehydroalanine, γ-amino butyric acid, and derivatives thereof wherein the amine nitrogen has been mono- or di-alkylated.

The term “amino” refers to a —NH₂ group;

The term “alkyl,” as a group, refers to a straight or branched hydrocarbon chain containing the specified number of carbon atoms. When the term “alkyl” is used without reference to a number of carbon atoms, it is to be understood to refer to a C₁-C₁₀ alkyl. For example, C₁₋₁₀ alkyl means a straight or branched alkyl containing at least 1, and at most 10, carbon atoms. Examples of “alkyl” as used herein include, but are not limited to, methyl, ethyl, n-propyl, n-butyl, n-pentyl, isobutyl, isopropyl, t-butyl, hexyl, heptyl, octyl, nonyl and decyl.

The term “substituted alkyl” as used herein denotes alkyl radicals wherein at least one hydrogen is replaced by one more substituents such as, but not limited to, hydroxy, carboxyl, alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., —C(O)O—R where R is an alkyl such as methyl) and acyloxyester (e.g., —OC(O)—R where R is an alkyl such as methyl). The definition pertains whether the term is applied to a substituent itself or to a substituent of a substituent.

The term “heterocycle” refers to a stable 3- to 15-membered ring radical which consists of carbon atoms and from one to five heteroatoms selected from nitrogen, phosphorus, oxygen and sulphur.

The term “cycloalkyl” group as used herein refers to a non-aromatic monocyclic hydrocarbon ring of 3 to 8 carbon atoms such as, for example, cyclopropyl, cyclobutyl, cyclopentyl, cyclohexyl or cycloheptyl.

The term “substituted cycloalkyl” as used herein denotes a cycloalkyl group further bearing one or more substituents as set forth herein, such as, but not limited to, hydroxy, carboxyl, alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., —C(O)O—R where R is an alkyl such as methyl) and acyloxyester (e.g., —OC(O)—R where R is an alkyl such as methyl). The definition pertains whether the term is applied to a substituent itself or to a substituent of a substituent.

The terms “keto” and “oxo” are synonymous and refer to the group ═O;

The terms “thioketo” and “thioxo” are synonymous and refer to the group ═S;

The term “carbonyl” refers to a group —C(═O);

The term “carboxyl” refers to a group —CO₂H and consists of a carbonyl and a hydroxyl group (More specifically, C(═O)OH);

The terms “carbamate group,” and “carbamate,” concern the group

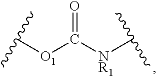

wherein the —O₁— is present in the unbound form of the opioid analgesic. Prodrug moieties described herein may be referred to based on their amino acid or peptide and the carbamate linkage. The amino acid or peptide in such a reference should be assumed to be bound via an amino terminus on the amino acid or peptide to the carbonyl linker and the opioid analgesic, unless otherwise specified.

For example, val carbamate (valine carbamate) has the formula

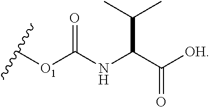

For a peptide, such as tyr-val carbamate, it should be assumed unless otherwise specified that the leftmost amino acid in the peptide is at the amino terminus of the peptide, and is bound via the carbonyl linker to the opioid analgesic to form the carbamate prodrug.

The term “thiocarbamate group,” and “thiocarbamate” refer to the group

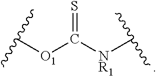

For example, val thiocarbamate (valine thicarbamate) has the formula

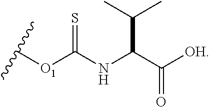

The abbreviation “MVC,” refers to the prodrug meptazinol valine carbamate.

The term “carrier” refers to a diluent, excipient, and/or vehicle with which an active compound is administered. The pharmaceutical compositions of the invention may contain one or a combination of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil and sesame oil. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18^{th} Edition.

The phrase “pharmaceutically acceptable” refers to molecular entities and compositions that are generally regarded as safe. In particular, pharmaceutically acceptable carriers used in the practice of this invention are physiologically tolerable and do not typically produce an allergic or similar untoward reaction (for example, gastric upset, dizziness) when administered to a subject. Preferably, as used herein, the term “pharmaceutically acceptable” means approved by a regulatory agency of the appropriate governmental agency or listed in the U.S. Pharmacopoeia or other generally recognized pharmacopoeia for use in animals, and more particularly in humans.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The term “treating” includes: (1) preventing or delaying the appearance of clinical symptoms of the state, disorder or condition developing in an animal that may be afflicted with or predisposed to the state, disorder or condition but does not yet experience or display clinical or subclinical symptoms of the state, disorder or condition; (2) inhibiting the state, disorder or condition (i.e., arresting, reducing or delaying the development of the disease, or a relapse thereof in case of maintenance treatment, of at least one clinical or subclinical symptom thereof); and/or (3) relieving the condition (i.e., causing regression of the state, disorder or condition or at least one of its clinical or subclinical symptoms). The benefit to a subject to be treated is either statistically significant or at least perceptible to the subject or to the physician.

“Effective amount” means an amount of an opioid prodrug used in the present invention sufficient to result in the desired therapeutic response. The therapeutic response can be any response that a user or clinician will recognize as an effective response to the therapy. The therapeutic response will generally be an analgesic response affording pain relief. It is further within the skill of one of ordinary skill in the art to determine an appropriate treatment duration, appropriate doses, and any potential combination treatments, based upon an evaluation of therapeutic response.

The term “subject” includes humans and other mammals, such as domestic animals (e.g., dogs and cats).

The term “salts” can include acid addition salts or addition salts of free bases. Suitable pharmaceutically acceptable salts (for example, of the carboxyl terminus of the amino acid or peptide) include, but are not limited to, metal salts such as sodium potassium and cesium salts; alkaline earth metal salts such as calcium and magnesium salts; organic amine salts such as triethylamine, guanidine and N-substituted guanidine salts, acetamidine and N-substituted acetamidine, pyridine, picoline, ethanolamine, triethanolamine, dicyclohexylamine, and N,N′-dibenzylethylenediamine salts. Pharmaceutically acceptable salts (of basic nitrogen centers) include, but are not limited to inorganic acid salts such as the hydrochloride, hydrobromide, sulfate, phosphate; organic acid salts such as trifluoroacetate and maleate salts; sulfonates such as methanesulfonate, ethanesulfonate, benzenesulfonate, p-toluenesulfonate, camphor sulfonate and naphthalenesulfonate; and amino acid salts such as arginate, gluconate, galacturonate, alaninate, asparginate and glutamate salts (see, for example, Berge, et al. “Pharmaceutical Salts,” J. Pharma. Sci. 1977; 66:1).

The term “active ingredient,” unless specifically indicated, is to be understood as referring to the opioid portion of the prodrug, described herein.

Compounds of the Invention

Without wishing to be bound to any theory, opioids may interact with the receptors within the gut wall, which can lead to adverse GI side effects (Holzer (2007). Expert Opin. Investig. Drugs 16, 181-194; Udeh and Goldman, US National Formulary 2005).

Additionally, concurrent oral administration of the locally acting (within the gut lumen) narcotic antagonist alvimopan with various opioids has been shown to markedly reduce the adverse GI effects of the latter, in terms of constipation, nausea and vomiting (Linn and Steinbrook (2007). Tech. in Regional Anaes. and Pain Mangmt 11, 27-32). Furthermore, a recently introduced combination product (Targin®) comprising oxycodone and the largely GI confined mu (μ) receptor antagonist naloxone, in a 2:1 ratio, has been shown to be associated with a reduced constipatory effect. A ˜50% reduction in the adverse effects on bowel function was reported compared with oxycodone used alone (Meissner et al. (2009). Eur. J. Pain 13, 56-64).

Therefore, without being bound to any particular theory, the prodrugs of the present invention reduce opioid induced adverse GI side effects by avoiding or minimizing interaction with opioid or other relevant receptors within the gut lumen. Subsequent to absorption, the active analgesic is regenerated (i.e., the prodrug is dissociated to form the unbound opioid analgesic) to effect the desired analgesic response. One advantage of the prodrugs of the present invention is that they eliminate the need for co-administration of medicaments to reverse the adverse GI effects of opioids such as anti-emetic agents, or narcotic antagonists such as alvimopan or naloxone.

In one embodiment, the present invention is directed to an opioid prodrug of Formula I

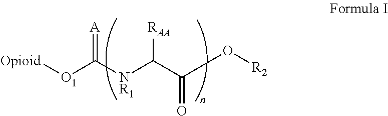

or a pharmaceutically acceptable salt thereof, wherein

O₁ is a hydroxylic oxygen present in the unbound opioid molecule,

A is selected from O and S,

each occurrence of R₁ is independently hydrogen, alkyl or substituted alkyl,

R₂ is a C₁-C₄ alkyl, an amino acid (e.g., serine (—CH₂CH(NH₂)COOH)), a substituted phenyl group (e.g., substituted with a carboxyl group, such as 2-COOH-phenyl), or a substituted alkyl group,

n is an integer from 1 to 9 (e.g., n can be 1),

each occurrence of R_{AA} is independently a proteinogenic or non-proteinogenic amino acid side chain, and

the opioid is selected from butorphanol, buprenorphine, codeine, dezocine, dihydrocodeine, hydromorphone, levorphanol, meptazinol, morphine, nalbuphine, oxycodone, oxymorphone, and pentazocine, or active metabolites thereof (e.g., ethyl-hydroxylated meptazinol (3-[3-(2-Hydroxy-ethyl)-1-methyl-perhydro-azepin-3-yl]-phenol), ethyl-carboxylated meptazinol (3-[3-(2-carboxy-ethyl)-1-methyl-perhydro-azepin-3-yl]-phenol), des-methyl meptazinol, 2-oxomeptazinol and 7-oxomeptazinol).

In one Formula I embodiment, R₂ is selected from t-butyl, isopropyl, ethyl, methyl,

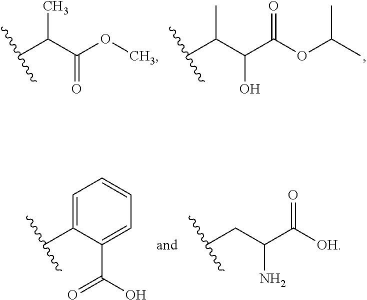

In a further Formula I embodiment, n is 1. In a further Formula I embodiment, R_{AA} is a proteinogenic amino acid side chain.

In another embodiment, R₂ is not t-butyl. In another embodiment, R₂ is methyl, ethyl, or isopropyl. R₂ is

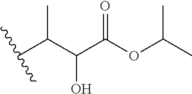

in another Formula I embodiment. In a further embodiment, n is 1 or 2. In still a further Formula I embodiment, R_{AA} is limited to proteinogenic amino acid side chains.

In one Formula I embodiment, the carbamate or thiocarbamate prodrug of the present invention is a lactone of Formula I.

In some Formula I embodiments, n is 1, 2, 3, 4 or 5.

In a preferred Formula I embodiment, the prodrug moiety of the compound of Formula I has one, two or three amino acids (i.e., n=1, 2 or 3), while R₂ is H.

In another Formula I embodiment, n is 2.

In yet another Formula I embodiment, n is 1 or 2 and each occurrence of R_{AA} is independently a proteinogenic amino acid side chain.

In yet another Formula I embodiment, n is 1 or 2 and at least one occurrence of R_{AA} is a non-proteinogenic amino acid side chain.

The present invention is also directed to a pharmaceutical composition comprising one or more of the opioid prodrugs of Formula I, and one or more pharmaceutically acceptable excipients.

In one Formula I embodiment, the

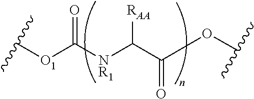

moiety of the present invention is selected from valine carbamate, L-met carbamate, 2-amino-butyric acid carbamate, ala carbamate, phe carbamate, ile carbamate, 2-amino acetic acid carbamate, leu carbamate, ala-ala carbamate, val-val carbamate, tyr-gly carbamate, val-tyr carbamate, tyr-val carbamate and val-gly carbamate.

In another embodiment, the present invention is directed to an opioid prodrug of Formula II:

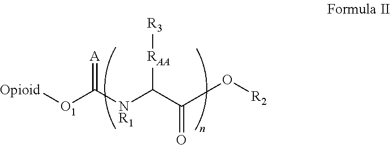

or a pharmaceutically acceptable salt thereof, wherein,

O₁ is a hydroxylic oxygen present in the unbound opioid molecule,

A is selected from O and S,

R₁ is H, alkyl or substituted alkyl,

R₂ is selected from H, cycloalkyl, aryl, substituted cycloalkyl, alkyl, substituted alkyl group and an opioid,

If R₂ is an opioid, —O— is a hydroxylic oxygen present in the unbound opioid,

n is an integer from 1 to 9 (e.g., n can be 1),

R_{AA} is a proteinogenic or non-proteinogenic amino acid side chain, and each occurrence of R_{AA} can be the same or different,

each occurrence of R₃ is independently absent or an amino acid (e.g., cysteine), each amino acid R₃ is bonded to R_{AA} via a side chain, N-terminus or C-terminus of the amino acid,

the opioid is selected from butorphanol, buprenorphine, codeine, dezocine, dihydrocodeine, hydromorphone, levorphanol, meptazinol, morphine, nalbuphine, oxycodone, oxymorphone, and pentazocine, or active metabolites thereof (e.g., ethyl-hydroxylated meptazinol (3-[3-(2-Hydroxy-ethyl)-1-methyl-perhydro-azepin-3-yl]-phenol), ethyl-carboxylated meptazinol (3-[3-(2-carboxy-ethyl)-1-methyl-perhydro-azepin-3-yl]-phenol), des-methyl meptazinol, 2-oxomeptazinol and 7-oxomeptazinol).

In one Formula II embodiment, the opioid is meptazinol, R₂ is an opioid, R₃ is absent and n is 1. In a further embodiment, R_{AA} is a valine side chain and R₂ is meptazinol.

In one Formula II embodiment, R₂ is selected from t-butyl, isopropyl, ethyl, methyl,

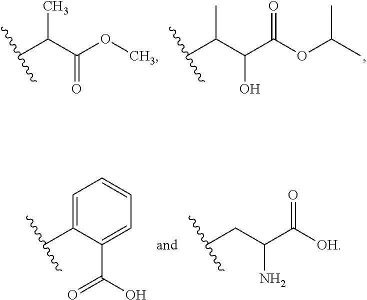

In a further Formula II embodiment, n is 1. In a further Formula II embodiment, R_{AA} is a proteinogenic amino acid side chain.

R₂ is

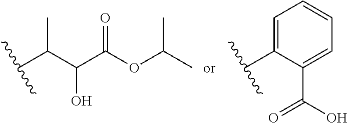

in another Formula II embodiment.

In one Formula II embodiment, the opioid is selected from buprenorphine, morphine, nalbuphine and oxycodone. In a further Formula II embodiment, n is 1, 2 or 3 and at least one occurrence of R_{AA} is a proteinogenic amino acid side chain.

In one embodiment, the carbamate or thiocarbamate prodrug of the present invention is a lactone of Formula II.

n is 1, R₃ is cysteine and R_{AA} is a cysteine side chain in one Formula II embodiment. In a further Formula II embodiment, R₂ is H, methyl, isopropyl,

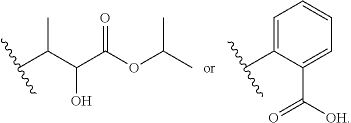

In some Formula II embodiments, n is 1, 2, 3, 4 or 5.

In a preferred Formula II embodiment, the prodrug moiety of the compound of Formula II has one, two or three amino acids (i.e., n=1, 2 or 3), while R₂ is H.

In another Formula II embodiment, n is 2. At least one occurrence of R_{AA} is a proteinogenic amino acid side chain in a further Formula II embodiment.

In yet another Formula II embodiment, R_{AA} is

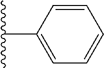

and n is 1. In a further Formula II embodiment, R₂ is H and R₃ is absent. In still a further Formula II embodiment, the opioid is selected from buprenorphine, codeine, dihydrocodeine, hydromorphone, meptazinol, morphine, nalbuphine, oxycodone and oxymorphone,

In yet another Formula II embodiment, R_{AA} is

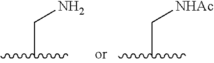

and n is 1. In a further Formula II embodiment, R₂ is H and R₃ is absent.

In yet another Formula II embodiment, R_{AA} is

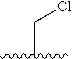

and n is 1. In a further Formula II embodiment, R₂ is H and R₃ is absent.

In yet another Formula II embodiment, R_{AA} is

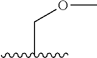

and n is 1. In a further Formula II embodiment, R₂ is H and R₃ is absent.

The present invention is also directed to a pharmaceutical composition comprising one or more of the opioid prodrugs of Formula II, and one or more pharmaceutically acceptable excipients.

In another embodiment, the present invention is directed to compounds of Formula III,

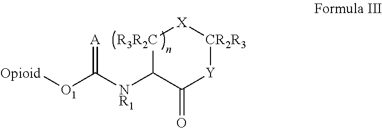

or a pharmaceutically acceptable salt thereof, wherein,

A and Y are independently selected from O and S,

X is absent or selected from O and S,

O₁ is a hydroxylic oxygen present in the unbound opioid molecule,

R₁ is H, alkyl or substituted alkyl,

R₂ and R₃ are independently selected from H, aryl, alkyl and substituted alkyl group,

n is an integer from 1 to 4 (e.g., n can be 1), and

the opioid is selected from butorphanol, buprenorphine, codeine, dezocine, dihydrocodeine, hydromorphone, levorphanol, meptazinol, morphine, nalbuphine, oxycodone, oxymorphone, and pentazocine, or active metabolites thereof (e.g., ethyl-hydroxylated meptazinol (3-[3-(2-Hydroxy-ethyl)-1-methyl-perhydro-azepin-3-yl]-phenol), ethyl-carboxylated meptazinol (3-[3-(2-carboxy-ethyl)-1-methyl-perhydro-azepin-3-yl]-phenol), des-methyl meptazinol, 2-oxomeptazinol and 7-oxomeptazinol).

In a further Formula III embodiment, the opioid is an active metabolite of meptazinol selected from ethyl-hydroxylated meptazinol (3-[3-(2-Hydroxy-ethyl)-1-methyl-perhydro-azepin-3-yl]-phenol), ethyl-carboxylated meptazinol (3-[3-(2-carboxy-ethyl)-1-methyl-perhydro-azepin-3-yl]-phenol), des-methyl meptazinol, 2-oxomeptazinol and 7-oxomeptazinol.

In one Formula III embodiment, n is 1, X is S and A is O. Y is O in a further Formula III embodiment. At least one occurrence of both R₂ and R₃ are methyl in a further embodiment.

In one Formula III embodiment, n is 1, X is O and A is O. Y is O in a further Formula III embodiment. At least one occurrence of both R₂ and R₃ are methyl in a further embodiment.

In one Formula III embodiment, n is 2, X is S and A is O. Y is O in a further Formula III embodiment. At least one occurrence of both R₂ and R₃ are methyl in a further embodiment.

In one Formula III embodiment, n is 2, X is O and A is O. Y is O in a further Formula III embodiment. At least one occurrence of both R₂ and R₃ are methyl in a further embodiment.

In one Formula III embodiment, R₂ and R₃ between the X and Y atoms are both methyl. In a further Formula III embodiment, n is 1. In still a further Formula III embodiment, X is O and the additional R₂ group is methyl, while R₃ is H.

In one Formula III embodiment, R₂ and R₃ between the X and Y atoms are both methyl. In a further Formula III embodiment, n is 1. In still a further Formula III embodiment, X is S and the additional R₂ group is methyl, while R₃ is H.

The present invention is also directed to a pharmaceutical composition comprising one or more of the opioid prodrugs of Formula III, and one or more pharmaceutically acceptable excipients.

In one embodiment, the opioid prodrugs of the present invention are directed to compounds of Formula IV:

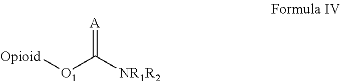

or a pharmaceutically acceptable salt thereof, wherein,

O₁ is a hydroxylic oxygen present in the unbound opioid molecule,

A is selected from O and S,

R₁ and R₂ are independently selected from H, aryl, alkyl and substituted alkyl, and

the opioid is selected from butorphanol, buprenorphine, codeine, dezocine, dihydrocodeine, hydromorphone, levorphanol, meptazinol, morphine, nalbuphine, oxycodone, oxymorphone, and pentazocine, or active metabolites thereof (e.g., ethyl-hydroxylated meptazinol (3-[3-(2-Hydroxy-ethyl)-1-methyl-perhydro-azepin-3-yl]-phenol), ethyl-carboxylated meptazinol (3-[3-(2-carboxy-ethyl)-1-methyl-perhydro-azepin-3-yl]-phenol), des-methyl meptazinol, 2-oxomeptazinol and 7-oxomeptazinol).

In a further Formula IV embodiment, the opioid is an active metabolite of meptazinol selected from ethyl-hydroxylated meptazinol (3-[3-(2-Hydroxy-ethyl)-1-methyl-perhydro-azepin-3-yl]-phenol), ethyl-carboxylated meptazinol (3-[3-(2-carboxy-ethyl)-1-methyl-perhydro-azepin-3-yl]-phenol), des-methyl meptazinol, 2-oxomeptazinol and 7-oxomeptazinol.

In one Formula IV embodiment, R₁ and R₂ are selected from propyl and butyl. In a further Formula IV embodiment, R₁ and R₂ are both propyl.

In one Formula IV embodiment, R₁ and R₂ are selected from hydrogen, methyl, propyl and butyl. In a further Formula IV embodiment, R₁ is hydrogen and R₂ is propyl.

In one Formula IV embodiment, R₁ and R₂ are selected from hydrogen, methyl, propyl and butyl. In a further Formula IV embodiment, R₁ is hydrogen and R₂ is butyl.

The present invention is also directed to a pharmaceutical composition comprising one or more of the opioid prodrugs of Formula IV, and one or more pharmaceutically acceptable excipients.

The opioid prodrugs of the present invention are also directed to compounds of Formula V:

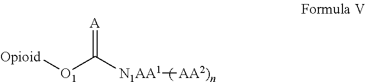

or a pharmaceutically acceptable salt thereof, wherein,

O₁ is a hydroxylic oxygen present in the unbound opioid molecule,

A is selected from O and S,

AA¹ is selected from a proteinogenic amino acid, a β-amino acid (e.g., β-alanine) and pyroglutamic acid,

AA² is an α- or β-amino acid (e.g., valine),

n is an integer from 0 to 9;

N₁ is a nitrogen atom present in the first AA, and

the opioid is selected from butorphanol, buprenorphine, codeine, dezocine, dihydrocodeine, hydromorphone, levorphanol, meptazinol, morphine, nalbuphine, oxycodone, oxymorphone, and pentazocine, or active metabolites thereof (e.g., ethyl-hydroxylated meptazinol (3-[3-(2-Hydroxy-ethyl)-1-methyl-perhydro-azepin-3-yl]-phenol), ethyl-carboxylated meptazinol (3-[3-(2-carboxy-ethyl)-1-methyl-perhydro-azepin-3-yl]-phenol), des-methyl meptazinol, 2-oxomeptazinol and 7-oxomeptazinol).

In a further Formula V embodiment, the opioid is an active metabolite of meptazinol selected from ethyl-hydroxylated meptazinol (3-[3-(2-Hydroxy-ethyl)-1-methyl-perhydro-azepin-3-yl]-phenol), ethyl-carboxylated meptazinol (3-[3-(2-carboxy-ethyl)-1-methyl-perhydro-azepin-3-yl]-phenol), des-methyl meptazinol, 2-oxomeptazinol and 7-oxomeptazinol.

In one Formula V embodiment, N₁ is the nitrogen atom of β-alanine. In a further Formula V embodiment, the opioid is selected from buprenorphine, codeine, dihydrocodeine, hydromorphone, meptazinol, morphine, nalbuphine, oxycodone and oxymorphone.

In one Formula V embodiment, N₁ is the nitrogen atom in a lysine side chain. In a further Formula V embodiment, n is 1 and the N-terminus of the lysine is bonded to valine. In still a further Formula V embodiment, the opioid is selected from buprenorphine, codeine, dihydrocodeine, hydromorphone, meptazinol, morphine, nalbuphine, oxycodone and oxymorphone.

In one Formula V embodiment, N₁ is the nitrogen atom of pyroglutamate and n is 0. In a further Formula V embodiment, the opioid is selected from buprenorphine, codeine, dihydrocodeine, hydromorphone, meptazinol, morphine, nalbuphine, oxycodone and oxymorphone.

In one embodiment, the opioid prodrugs of the present invention are directed to compounds of Formula V(A):

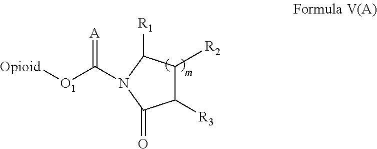

or a pharmaceutically acceptable salt thereof, wherein,

O₁ is a hydroxylic oxygen present in the unbound opioid molecule,

A is selected from O and S,

R₁, R₂ and R₃ are independently selected from hydrogen, aryl, alkyl, substituted alkyl group and carboxyl, and at least one occurrence of R₁, R₂ and R₃ is carboxyl,

m is an integer from 1 to 3; and

the opioid is selected from butorphanol, buprenorphine, codeine, dezocine, dihydrocodeine, hydromorphone, levorphanol, meptazinol, morphine, nalbuphine, oxycodone, oxymorphone, and pentazocine, or active metabolites thereof (e.g., ethyl-hydroxylated meptazinol (3-[3-(2-Hydroxy-ethyl)-1-methyl-perhydro-azepin-3-yl]-phenol), ethyl-carboxylated meptazinol (3-[3-(2-carboxy-ethyl)-1-methyl-perhydro-azepin-3-yl]-phenol), des-methyl meptazinol, 2-oxomeptazinol and 7-oxomeptazinol).

In one Formula V(A) embodiment, m is 1. In a further Formula V(A) embodiment, A is O. In a further Formula V(A) embodiment, R₁ is carboxyl and R₂ and R₃ are both hydrogen. In still a further Formula V(A) embodiment, the opioid is selected from buprenorphine, codeine, dihydrocodeine, hydromorphone, meptazinol, morphine, nalbuphine, oxycodone and oxymorphone.

In one Formula V(A) embodiment, m is 1. In a further Formula V(A) embodiment, A is S. In a further Formula V(A) embodiment, R₁ is carboxyl and R₂ and R₃ are both hydrogen. In still a further Formula V(A) embodiment, the opioid is selected from buprenorphine, codeine, dihydrocodeine, hydromorphone, meptazinol, morphine, nalbuphine, oxycodone and oxymorphone.

In one Formula V(A) embodiment, m is 2. In a further Formula V(A) embodiment, A is O. In a further Formula V(A) embodiment, R₁ is carboxyl and R₂ and R₃ are both hydrogen. In still a further Formula V(A) embodiment, the opioid is selected from buprenorphine, codeine, dihydrocodeine, hydromorphone, meptazinol, morphine, nalbuphine, oxycodone and oxymorphone.

In one Formula V(A) embodiment, m is 2. In a further Formula V(A) embodiment, A is S. In a further Formula V(A) embodiment, R₁ is carboxyl and R₂ and R₃ are both hydrogen. In still a further Formula V(A) embodiment, the opioid is selected from buprenorphine, codeine, dihydrocodeine, hydromorphone, meptazinol, morphine, nalbuphine, oxycodone and oxymorphone.

In one Formula V(A) embodiment, m is 3. In a further Formula V(A) embodiment, A is O. In a further Formula V(A) embodiment, R₁ is carboxyl and R₂ and R₃ are both hydrogen. In still a further Formula V(A) embodiment, the opioid is selected from buprenorphine, codeine, dihydrocodeine, hydromorphone, meptazinol, morphine, nalbuphine, oxycodone and oxymorphone.

In one Formula V(A) embodiment, m is 3. In a further Formula V(A) embodiment, A is S. In a further Formula V(A) embodiment, R₁ is carboxyl and R₂ and R₃ are both hydrogen. In still a further Formula V(A) embodiment, the opioid is selected from buprenorphine, codeine, dihydrocodeine, hydromorphone, meptazinol, morphine, nalbuphine, oxycodone and oxymorphone.

In one Formula V(A) embodiment, at least one carboxyl moiety of R₁, R₂ or R₃ is bound to an amino acid or peotide. In a further Formula V(A) embodiment, the amino acid bound to the at least one carboxyl moiety is valine. In still a further Formula V(A) embodiment, the opioid is selected from buprenorphine, codeine, dihydrocodeine, hydromorphone, meptazinol, morphine, nalbuphine, oxycodone and oxymorphone.

The present invention is also directed to a pharmaceutical composition comprising one or more of the opioid prodrugs of Formula V(A), and one or more pharmaceutically acceptable excipients.

In yet another embodiment, the present invention is directed to an opioid prodrug of Formula VI:

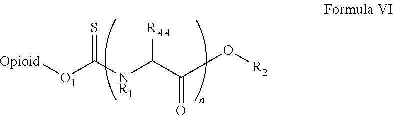

or a pharmaceutically acceptable salt thereof, wherein,

O₁ is a hydroxylic oxygen present in the unbound opioid molecule,

R₁ is selected from hydrogen, unsubstituted alkyl, substituted alkyl, cycloalkyl, and substituted cycloalkyl group,

R₂ is selected from hydrogen, unsubstituted alkyl, substituted alkyl, cycloalkyl, substituted cycloalkyl group, and an opioid, and if R₂ is an opioid, the —O— is a hydroxylic oxygen in the opioid,

n is an integer from 1 to 9,

each occurrence of R_{AA} is independently a proteinogenic or non-proteinogenic amino acid side chain (e.g., R_{AA} can be isopropyl), and

the opioid is selected from butorphanol, buprenorphine, codeine, dezocine, dihydrocodeine, hydromorphone, levorphanol, meptazinol, morphine, nalbuphine, oxycodone, oxymorphone, and pentazocine, or active metabolites thereof (e.g., ethyl-hydroxylated meptazinol (3-[3-(2-Hydroxy-ethyl)-1-methyl-perhydro-azepin-3-yl]phenol), ethyl-carboxylated meptazinol (3-[3-(2-carboxy-ethyl)-1-methyl-perhydro-azepin-3-yl]-phenol), des-methyl meptazinol, 2-oxomeptazinol and 7-oxomeptazinol).

R₂ in one Formula VI embodiment is hydrogen or C₁-C₄ alkyl.

In one Formula VI embodiment, R_{AA} is isopropyl and the carbon atom attached to R_{AA} is in the S configuration.

In one Formula VI embodiment, the opioid is meptazinol, R₂ is an opioid, and n is 1. In a further embodiment, R_{AA} is a valine side chain and R₂ is meptazinol.

In one Formula VI embodiment, R₂ is selected from t-butyl, isopropyl, ethyl, methyl,

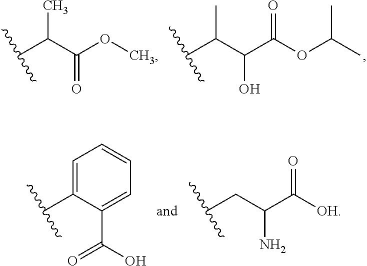

In a further Formula VI embodiment, n is 1. In a further Formula VI embodiment, R_{AA} is a proteinogenic amino acid side chain.

R₂ is

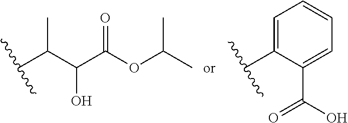

in another Formula VI embodiment.

In one Formula VI embodiment, the opioid is selected from buprenorphine, morphine, nalbuphine and oxycodone. In a further Formula VI embodiment, n is 1, 2 or 3 and at least one occurrence of R_{AA} is a proteinogenic amino acid side chain.

In one embodiment, the thiocarbamate prodrug is a lactone of Formula VI.

n is 1 in one Formula VI embodiment. In a further Formula VI embodiment, R₂ is H, methyl, isopropyl,

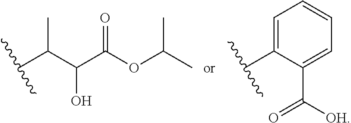

In some Formula VI embodiments, n is 1, 2, 3, 4 or 5.

In a preferred Formula VI embodiment, the prodrug moiety of the compound of Formula VI has one, two or three amino acids (i.e., n=1, 2 or 3), while R₂ is H.

In another Formula VI embodiment, n is 2. At least one occurrence of R_{AA} is a proteinogenic amino acid side chain in a further Formula VI embodiment.

In yet another Formula VI embodiment, R_{AA} is

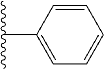

and n is 1. In a further Formula VI embodiment, and R₁ and R₂ are both H. In still a further Formula VI embodiment, the opioid is selected from buprenorphine, codeine, dihydrocodeine, hydromorphone, meptazinol, morphine, nalbuphine, oxycodone and oxymorphone,

In yet another Formula VI embodiment, R_{AA} is

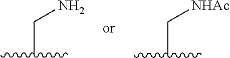

and n is 1. In a further Formula II embodiment, R₁ and R₂ are both H.

In yet another Formula VI embodiment, R_{AA} is

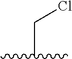

and n is 1. In a further Formula II embodiment, R₁ and R₂ are both H.

In yet another Formula VI embodiment, R_{AA} is

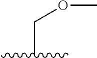

and n is 1. In a further Formula II embodiment, R₁ and R₂ are both H.

In a preferred Formula I embodiment, the prodrug moiety of the compound of Formula VI has one, two or three amino acids (i.e., n=1, 2 or 3), while R₂ is H.

In another Formula VI embodiment, n is 2.

In yet another Formula VI embodiment, n is 1 or 2 and each occurrence of R_{AA} is independently a proteinogenic amino acid side chain.

In yet another Formula VI embodiment, n is 1 or 2 and at least one occurrence of R_{AA} is a non-proteinogenic amino acid side chain.

In yet another embodiment, the present invention is directed to an opioid prodrug of Formula VII:

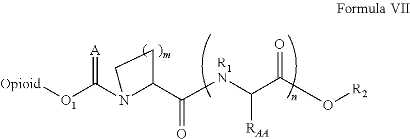

or a pharmaceutically acceptable salt thereof, wherein,

O₁ is a hydroxylic oxygen present in the unbound opioid molecule,

A is selected from O and S,

each occurrence of R₁ is independently hydrogen, alkyl or substituted alkyl,

m is an integer from 1 to 4 and n is an integer from 0 to 9,

R₂ is selected from hydrogen, C₁-C₄ alkyl, an amino acid (e.g., serine (—CH₂CH(NH₂)COOH)), or a substituted phenyl group (e.g., substituted with a carboxyl group, such as 2-COOH-phenyl) and an opioid,

If R₂ is an opioid, —O— is a hydroxylic oxygen present in the unbound opioid,

each occurrence of R_{AA} is independently a proteinogenic or non-proteinogenic amino acid side chain, and

the opioid is selected from butorphanol, buprenorphine, codeine, dezocine, dihydrocodeine, hydromorphone, levorphanol, meptazinol, morphine, nalbuphine, oxycodone, oxymorphone, and pentazocine, or active metabolites thereof (e.g., ethyl-hydroxylated meptazinol (3-[3-(2-Hydroxy-ethyl)-1-methyl-perhydro-azepin-3-yl]-phenol), (3-[3-(2-carboxy-ethyl)-1-methyl-perhydro-azepin-3-yl]-phenol), des-methyl meptazinol, 2-oxomeptazinol and 7-oxomeptazinol).

In one Formula VII embodiment, R₂ is not hydrogen.

In one Formula VII embodiment, A is 0, m is 2, n is 0, and R₂ is hydrogen. In this embodiment, the prodrug moiety is proline carbamate.

In one Formula VII embodiment, m is 1 and A is O. In a further Formula VII embodiment, n is 0, 1 or 2. In a further Formula VII embodiment, at least one R_{AA} is a proteinogenic amino acid side chain. In still a further embodiment, the at least one R_{AA} is a proteinogenic amino acid side chain is selected from valine, leucine and isoleucine.

In one Formula VII embodiment, m is 1 and A is S. In a further Formula VII embodiment, n is 0, 1 or 2. In a further Formula VII embodiment, at least one R_{AA} is a proteinogenic amino acid side chain. In still a further embodiment, the at least one R_{AA} is a proteinogenic amino acid side chain is selected from valine, leucine and isoleucine.

In one Formula VII embodiment, m is 2 and A is O. In a further Formula VII embodiment, n is 0, 1 or 2. In a further Formula VII embodiment, at least one R_{AA} is a proteinogenic amino acid side chain. In still a further embodiment, the at least one R_{AA} is a proteinogenic amino acid side chain is selected from valine, leucine and isoleucine.

In one Formula VII embodiment, m is 2 and A is S. In a further Formula VII embodiment, n is 0, 1 or 2. In a further Formula VII embodiment, at least one R_{AA} is a proteinogenic amino acid side chain. In still a further embodiment, the at least one R_{AA} is a proteinogenic amino acid side chain is selected from valine, leucine and isoleucine.

In one Formula VII embodiment, m is 3 and A is O. In a further Formula VII embodiment, n is 0, 1 or 2. In a further Formula VII embodiment, at least one R_{AA} is a proteinogenic amino acid side chain. In still a further embodiment, the at least one R_{AA} is a proteinogenic amino acid side chain is selected from valine, leucine and isoleucine.

In one Formula VII embodiment, m is 3 and A is S. In a further Formula VII embodiment, n is 0, 1 or 2. In a further Formula VII embodiment, at least one R_{AA} is a proteinogenic amino acid side chain. In still a further embodiment, the at least one R_{AA} is a proteinogenic amino acid side chain is selected from valine, leucine and isoleucine.

In one Formula VII embodiment, m is 4 and A is O. In a further Formula VII embodiment, n is 0, 1 or 2. In a further Formula VII embodiment, at least one R_{AA} is a proteinogenic amino acid side chain. In still a further embodiment, the at least one R_{AA} is a proteinogenic amino acid side chain is selected from valine, leucine and isoleucine.

In one Formula VII embodiment, m is 4 and A is S. In a further Formula VII embodiment, n is 0, 1 or 2. In a further Formula VII embodiment, at least one R_{AA} is a proteinogenic amino acid side chain. In still a further embodiment, the at least one R_{AA} is a proteinogenic amino acid side chain is selected from valine, leucine and isoleucine.

In one Formula VII embodiment, the opioid is meptazinol, R₂ is an opioid, and n is 1. In a further embodiment, R_{AA} is a valine side chain and R₂ is meptazinol.

In one Formula VII embodiment, R₂ is selected from t-butyl, isopropyl, ethyl, methyl,

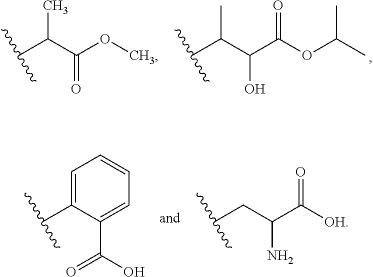

In a further Formula II embodiment, n is 1. In a further Formula VII embodiment, R_{AA} is a proteinogenic amino acid side chain.

R₂ is

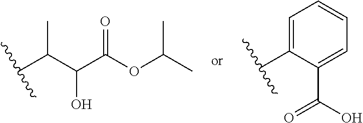

in another Formula VII embodiment.

In one Formula VII embodiment, the opioid is selected from buprenorphine, morphine, nalbuphine and oxycodone. In a further Formula VII embodiment, n is 1, 2 or 3 and at least one occurrence of R_{AA} is a proteinogenic amino acid side chain. In still a further embodiment, the at least one R_{AA} is a proteinogenic amino acid side chain is selected from valine, leucine and isoleucine.

In one Formula VII embodiment, the prodrug is a lactone of Formula VII.

n is 1 in one Formula VII embodiment. In a further Formula VII embodiment, R₂ is H, methyl, isopropyl,

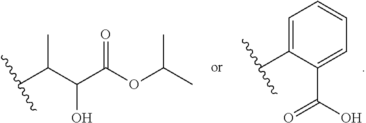

In a preferred Formula VII embodiment, the prodrug moiety of the compound of Formula VII has one, two or three amino acids, while R₂ is H.

In another Formula VII embodiment, n is 2. At least one occurrence of R_{AA} is a proteinogenic amino acid side chain in a further Formula VII embodiment.

In yet another Formula VII embodiment, R_{AA} is

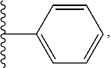

m is 1 or 2 and n is 1. In a further Formula VII embodiment, and R₁ and R₂ are both H. In still a further Formula VII embodiment, the opioid is selected from buprenorphine, codeine, dihydrocodeine, hydromorphone, meptazinol, morphine, nalbuphine, oxycodone and oxymorphone,

In yet another Formula VII embodiment, R_{AA} is

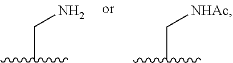

m is 1 or 2 and n is 1. In a further Formula VII embodiment, R₁ and R₂ are both H.

In yet another Formula VII embodiment, R_{AA} is

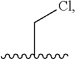

m is 1 or 2 and n is 1. In a further Formula VII embodiment, R₁ and R₂ are both H.

In yet another Formula VII embodiment, R_{AA} is

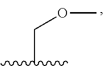

m is 1 or 2 and n is 1. In a further Formula VII embodiment, R₁ and R₂ are both H.

In a preferred Formula VII embodiment, the prodrug moiety of the compound of Formula VII has one, two or three amino acids (i.e., n=1, 2 or 3), while R₂ is H.

In another Formula VII embodiment, n is 2.

In yet another Formula VII embodiment, n is 1 or 2 and each occurrence of R_{AA} is independently a proteinogenic amino acid side chain.

In yet another Formula VII embodiment, n is 1 or 2 and at least one occurrence of R_{AA} is a non-proteinogenic amino acid side chain.

In yet another embodiment, the present invention is directed to an opioid prodrug of Formula VIII:

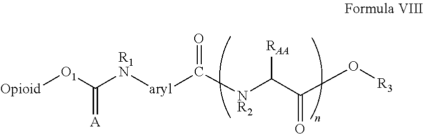

or a pharmaceutically acceptable salt thereof, wherein,

O₁ is a hydroxylic oxygen present in the unbound opioid molecule,

R₁ is selected from hydrogen, alkyl, substituted alkyl, cycloalkyl and substituted cycloalkyl group,

Each occurrence of R₂ is independently selected from hydrogen, alkyl, substituted alkyl, cycloalkyl, or substituted cycloalkyl group,

R₃ is selected from hydrogen, alkyl, substituted alkyl, cycloalkyl, substituted cycloalkyl group and an opioid,

If R₃ is an opioid, —O— is a hydroxylic oxygen present in the unbound opioid,

NR₁ and the carboxyl group immediately flanking the aryl group in Formula VIII can be a part of the aryl group,

n is an integer from 1 to 9,

each occurrence of R_{AA} is independently a proteinogenic or non-proteinogenic amino acid side chain (e.g., R_{AA} can be isopropyl) and

the opioid is selected from butorphanol, buprenorphine, codeine, dezocine, dihydrocodeine, hydromorphone, levorphanol, meptazinol, morphine, nalbuphine, oxycodone, oxymorphone, and pentazocine, or active metabolites thereof (e.g., ethyl-hydroxylated meptazinol (3-[3-(2-Hydroxy-ethyl)-1-methyl-perhydro-azepin-3-yl]-phenol), ethyl-carboxylated meptazinol (3-[3-(2-carboxy-ethyl)-1-methyl-perhydro-azepin-3-yl]-phenol), des-methyl meptazinol, 2-oxomeptazinol and 7-oxomeptazinol).

In a preferred Formula VIII embodiment, the

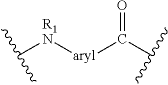

moiety is selected from

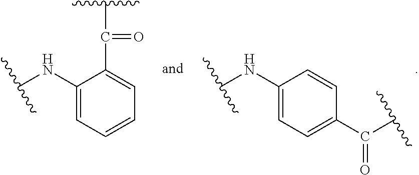

In one Formula VIII embodiment, the opioid is meptazinol, R₃ is an opioid, and n is 1. In a further embodiment, R_{AA} is a valine side chain, R₃ is meptazinol and the

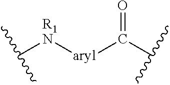

moiety is selected from

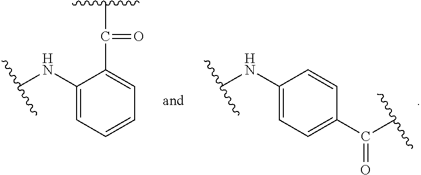

In one Formula VIII embodiment, the

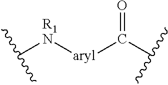

moiety is selected from

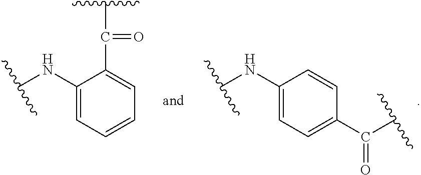

and R₃ is selected from t-butyl, isopropyl, ethyl, methyl,

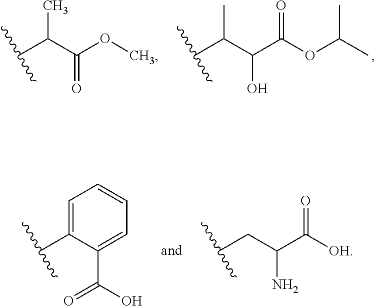

In a further Formula VIII embodiment, n is 1. In a further Formula VIII embodiment, R_{AA} is a proteinogenic amino acid side chain.

R₃ is

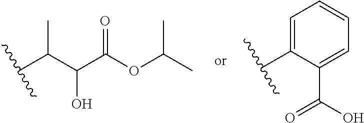

in another Formula VIII embodiment.

In one Formula VIII embodiment, the opioid is selected from buprenorphine, morphine, nalbuphine and oxycodone. In a further Formula VIII embodiment, In one Formula VIII embodiment, the

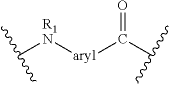

moiety is selected from

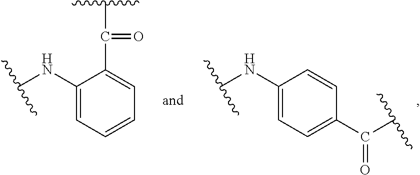

n is 1, 2 or 3 and at least one occurrence of R_{AA} is a proteinogenic amino acid side chain.

In one embodiment, the prodrug is a lactone of Formula VIII.

n is 1 in one Formula VIII embodiment. In a further Formula VIII embodiment, the

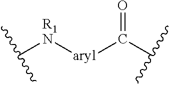

moiety is selected from

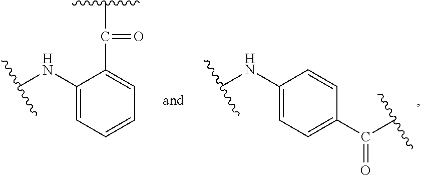

R₂ is H, methyl, isopropyl,

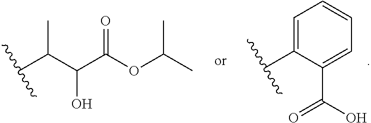

In a preferred Formula VIII embodiment, the prodrug moiety of the compound of Formula VIII has one, two or three amino acids, while R₂ and R₃ are both H.

In another Formula VIII embodiment, n is 2 and the

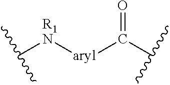

moiety is selected from

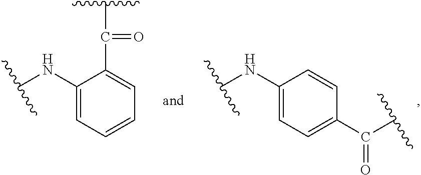

At least one occurrence of R_{AA} is a proteinogenic amino acid side chain in a further Formula VIII embodiment. In still a further embodiment, the at least one R_{AA} is a proteinogenic amino acid side chain is selected from valine, leucine and isoleucine.

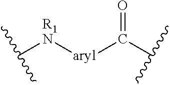

In yet another Formula VIII embodiment, the moiety is selected from

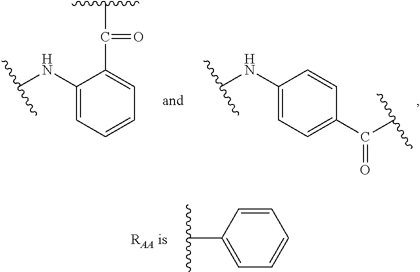

and n is 1. In a further Formula VIII embodiment, and R₁ and R₂ are both H. In still a further Formula VIII embodiment, the opioid is selected from buprenorphine, codeine, dihydrocodeine, hydromorphone, meptazinol, morphine, nalbuphine, oxycodone and oxymorphone.

In yet another Formula VIII embodiment, the

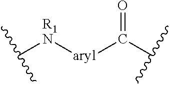

moiety is selected from

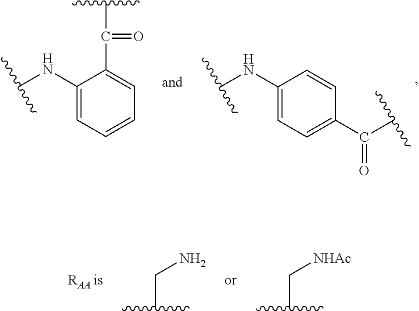

and n is 1. In a further Formula VIII embodiment, R₁ and R₂ are both H. In a further Formula VIII embodiment, the proteinogenic amino acid is selected from valine, isoleucine, alanine and leucine.

In yet another Formula VIII embodiment, the

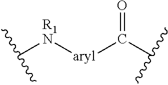

moiety is selected from

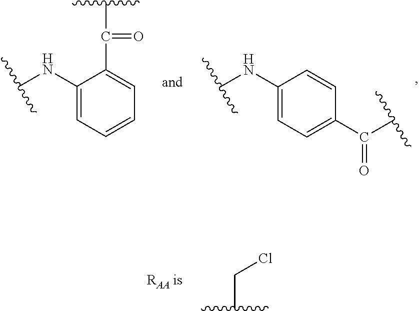

and n is 1. In a further Formula VIII embodiment, R₁ and R₂ are both H. In a further Formula VIII embodiment, the proteinogenic amino acid is selected from valine, isoleucine, alanine and leucine.

In yet another Formula VIII embodiment, the

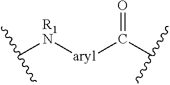

moiety is selected from

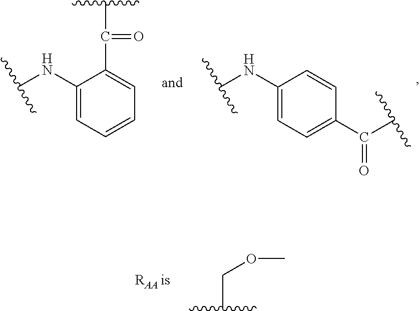

and n is 1. In a further Formula VIII embodiment, R₁ and R₂ are both H. In a further Formula VIII embodiment, the proteinogenic amino acid is selected from valine, isoleucine, alanine and leucine.

In a preferred Formula VIII embodiment, the prodrug moiety of the compound of Formula VIII has one, two or three amino acids (i.e., n=1, 2 or 3), while R₂ is H. In a further Formula VIII embodiment, the proteinogenic amino acid is selected from valine, isoleucine, alanine and leucine.

In another Formula VIII embodiment, n is 2. In a further Formula VIII embodiment, the proteinogenic amino acid is selected from valine, isoleucine, alanine and leucine.

In yet another Formula VIII embodiment, n is 1 or 2 and each occurrence of R_{AA} is independently a proteinogenic amino acid side chain. In a further Formula VIII embodiment, the proteinogenic amino acid is selected from valine, isoleucine, alanine and leucine.

In yet another Formula VIII embodiment, n is 1 or 2 and at least one occurrence of R_{AA} is a non-proteinogenic amino acid side chain.

Preferred prodrug moieties (e.g., the

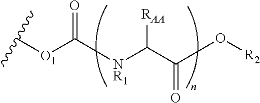

moiety) of the present invention include valine carbamate, leucine carbamate and isoleucine carbamate as single amino acid prodrug moities. Dipeptide moieties that are preferred include valine-valine carbamate, alanine-alanine carbamate and valine-glycine carbamate.

However, peptides comprising any of the proteinogenic amino acids, as well as non-proteinogenic amino acids, can be used in the present invention. Examples of non-proteinogenic amino acids are given above.

The 22 proteinogenic amino acids are given in Table 1 below.

TABLE 1
Proteinogenic Amino acids and Their
Abbreviations

| Amino acid | 3 letter code | 1-letter code |
|---|---|---|
| Alanine | ALA | A |
| Cysteine | CYS | C |
| Aspartic Acid | ASP | D |
| Glutamic Acid | GLU | E |
| Phenylalanine | PHE | F |
| Glycine | GLY | G |
| Histidine | HIS | H |
| Isoleucine | ILE | I |
| Lysine | LYS | K |
| Leucine | LEU | L |
| Methionine | MET | M |
| Asparagine | ASN | N |
| Proline | PRO | P |
| Glutamine | GLN | Q |
| Arginine | ARG | R |
| Serine | SER | S |
| Threonine | THR | T |
| Valine | VAL | V |
| Tryptophan | TRP | W |
| Tyrosine | TYR | Y |
| Selenocysteine | SEC | U |
| Pyrrolysine | PYL | O |

The amino acids employed in the opioid prodrugs for use with the present invention are preferably in the L configuration. The present invention also contemplates prodrugs of the invention comprised of amino acids in the D configuration, or mixtures of amino acids in the D and L configurations.

In another embodiment, the prodrug peptide moiety comprises a single amino acid, and when bound to the opioid analgesic, can be alanine carbamate, 2-amino-butyric acid carbamate, L-methionine carbamate, valine carbamate, or 2-amino acetic acid carbamate.

In other embodiments, the prodrug of the present invention comprises a dipeptide moiety, and can be tyrosine-valine carbamate, tyrosine-glycine-carbamate or valine-tyrosine carbamate.

The opioid analgesic of the present invention is conjugated to a peptide (which can be a single amino acid) through a carbamate linkage to the N-terminus of the peptide or amino acid. The peptide or amino acid can be conjugated to any free oxygen on the opioid analgesic.

In one embodiment, the peptide/amino acid (or multiple peptides or amino acids) can be bound to one of two (or both) possible locations in the opioid molecule. For example, morphine and dihydromorphine have hydroxyl groups at carbon 3 and carbon 6. A peptide or amino acid can be bound at either, or both of these positions. Carbamate linkages can be formed at either site, and upon peptide cleavage, the opioid will revert back to its original form. This general process is shown below in scheme 1, for three types of morphine prodrugs (i.e., with a peptide or amino acid linked at either or both the third and sixth carbons). For scheme 1, R₁, R₂ and R_{AA} are defined above, as provided for Formula I.

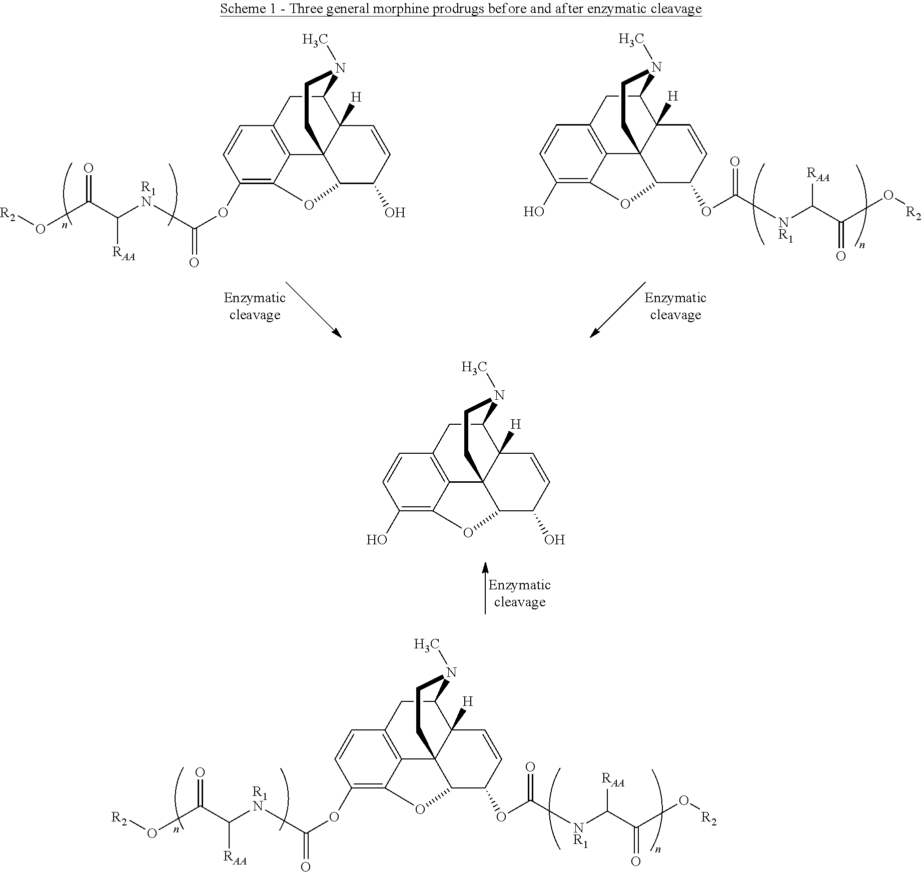

When a ketone is present in the opioid scaffold (e.g., the ketone at the 6 position of hydromorphone, and oxycodone), the ketone can be converted to its corresponding enolate and reacted with a modified peptide reactant (which can be a modified amino acid, see Examples) to form a prodrug. This linkage is depicted below in scheme 2, using hydromorphone as an example. Upon peptide cleavage, the prodrug will revert back to the original hydromorphone molecule, with the keto group present. Oxycodone can also have a peptide or amino acid linked at the 14 position, where a hydroxyl group is present. An oxycodone prodrug with a carbamate linkage at position 14 is shown in scheme 3, below. Additionally, the ketone in oxycodone can be converted to its corresponding enolate and reacted with a modified peptide reactant (which can be a modified amino acid, see Examples) to form a prodrug (not shown). For schemes 1-3, R₁, R₂, R_{AA} and n are defined as provided for Formula I.

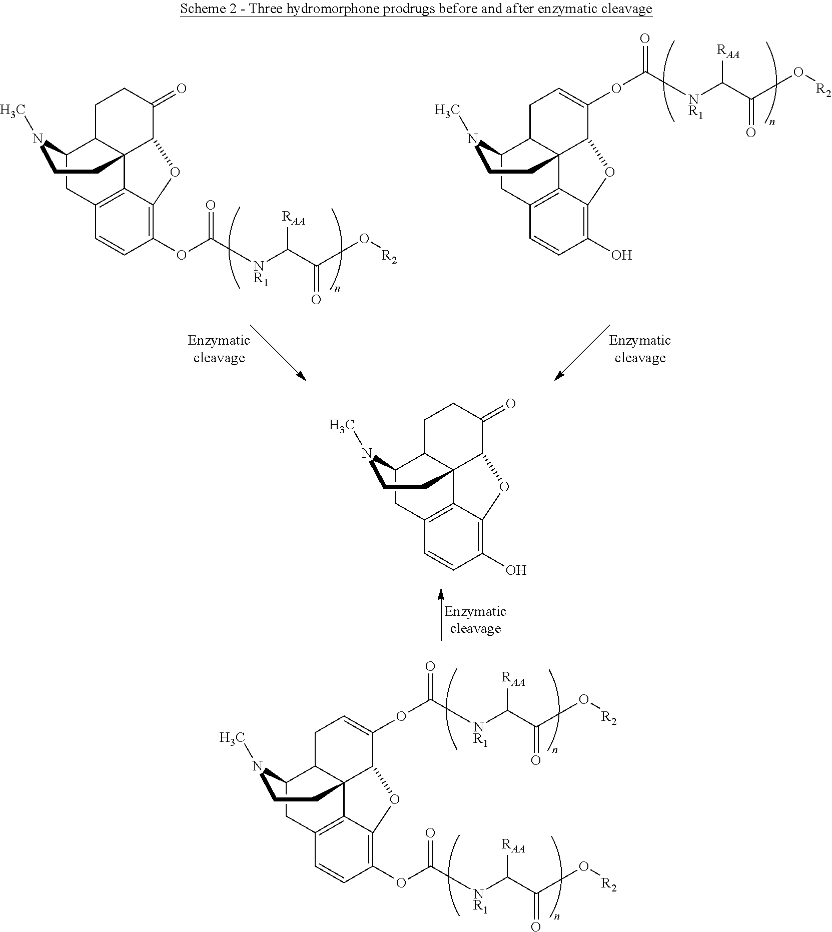

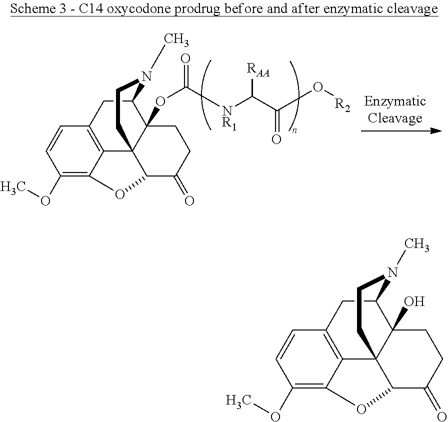

The following description pertains to meptazinol prodrugs. However, other opioids having a hydroxylic function can be readily substituted for meptazinol by those of ordinary skill in the art.

Meptazinol Compounds of the Present Invention

The novel meptazinol compounds of the present invention include prodrugs of Formula IX:

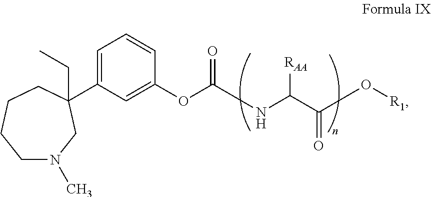

or a pharmaceutically acceptable salt thereof, wherein,

R₁ is selected from H, an alkyl group, a substituted alkyl group, meptazinol, an amino acid (e.g., serine (—CH₂CH(NH₂)COOH)), and a substituted phenyl group (e.g., substituted with a carboxyl group, such as 2-COOH-phenyl),

If R₁ is meptazinol, the —O— is the hydroxylic oxygen of meptazinol,

n is an integer from 1 to 9;

R_{AA} is a proteinogenic or non-proteinogenic amino acid side chain, and each occurrence of R_{AA} can be the same or different.

In one Formula IX embodiment, n is 1, 2 or 3.

In another Formula IX embodiment, R_{AA} is a valine side chain and R₁ is meptazinol.

In one Formula IX embodiment, R₂ is selected from t-butyl, isopropyl, ethyl, methyl,

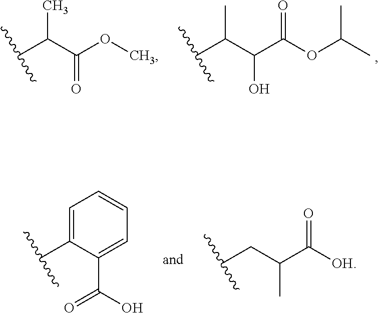

In a further Formula IX embodiment, R₂ is isopropyl.

In another Formula IX embodiment, R₂ is

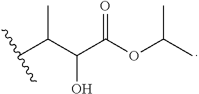

In a further Formula IX embodiment, n is 1 and R_{AA} is a proteinogenic amino acid side chain. In still a further embodiment, the proteinogenic amino acid side chain is selected from valine, leucine and isoleucine.

In a preferred Formula IX embodiment, n is 1, 2 or 3 and R₁ is H.

In another Formula IX embodiment, n is 1.

In yet another Formula IX embodiment, n is 2.

In yet another Formula IX embodiment, n is 1 or 2 and each occurrence of R_{AA} is independently a proteinogenic amino acid side chain.

In one Formula IX embodiment at least one of R_{AA} is valine and R₂ is isopropyl. In some Formula IX embodiments, n is 1, 2, 3, 4 or 5.

In a preferred Formula IX embodiment, the prodrug moiety of the compound of Formula I has one, two or three amino acids (i.e., n=1, 2 or 3), while R₂ is H.

In another Formula IX embodiment, n is 2.

In yet another Formula IX embodiment, n is 1 or 2 and at least one occurrence of R_{AA} is a non-proteinogenic amino acid side chain.

The present invention is also directed to a pharmaceutical composition comprising one or more of the opioid prodrugs of Formula IX, and one or more pharmaceutically acceptable excipients.

A preferred embodiment of the meptazinol prodrug of Formula IX is a prodrug wherein the amino acid side chain comprises a non-polar or an aliphatic amino acid, including the single amino acid prodrug meptazinol valine carbamate, shown below.

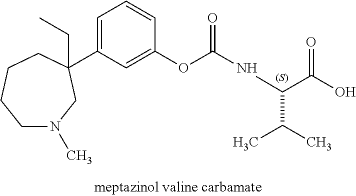

Single amino acid meptazinol carbamate prodrugs of the present invention include meptazinol-(S)-ile carbamate, meptazinol-(S)-leu carbamate, meptazinol-(S)-asp carbamate, meptazinol-(S)-met carbamate, meptazinol-(S)-his carbamate, meptazinol-(S)-phe carbamate and meptazinol-(S)-ser carbamate.

In a preferred meptazinol dipeptide embodiment (i.e., n is 2), the compound is selected from meptazinol-(S)-val-val carbamate, meptazinol-(S)-ile-ile and meptazinol-(S)-leu-leu.

In another embodiment, the meptazinol compounds of the present invention include prodrugs of Formula X:

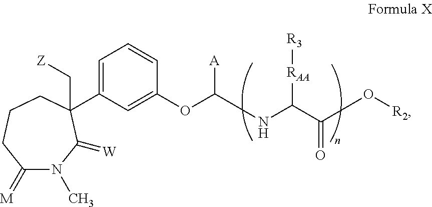

or a pharmaceutically acceptable salt thereof, wherein,

A is selected from O and S,

M and W are independently O or absent, and only one of M and W can be present on any one molecule,

Z is methyl, CH₂OH or COOH,

R₁ is H or methyl,

if Z is CH₂OH or COOH, M and W are both absent and R₁ is methyl,

if M or W is present, Z and R₁ are both methyl,

if R₁ is H, M and W are both absent while Z is methyl,

R₂ is selected from H, cycloalkyl, aryl, substituted cycloalkyl, alkyl, substituted alkyl group and an opioid,

If R₂ is an opioid, —O— is a hydroxylic oxygen present in the unbound opioid,

each occurrence of R₃ is independently absent or an amino acid (e.g., cysteine), each amino acid R₃ is bonded to R_{AA} via a side chain, N-terminus or C-terminus of the amino acid R₃,

n is an integer from 1 to 9, and

R_{AA} is a proteinogenic or non-proteinogenic amino acid side chain, and each occurrence of R_{AA} can be the same or different;

In one embodiment, the carbamate or thiocarbamate prodrug of the present invention is a lactone of Formula X.

In one Formula X embodiment, R₂ is meptazinol.

In one Formula X embodiment, M is O.

In one Formula X embodiment, W is O.

In one Formula X embodiment, R₂ is selected from t-butyl, isopropyl, ethyl, methyl,

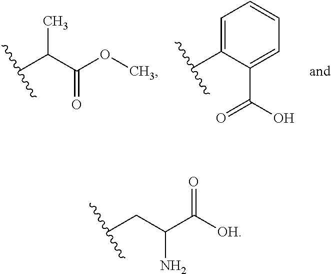

In a further Formula X embodiment, R₁ and Z are both methyl and M and W are both absent.

In one Formula X embodiment, the opioid is meptazinol, R₂ is an opioid, R₃ is absent and n is 1. In a further embodiment, R_{AA} is a valine side chain and R₂ is meptazinol. In a further Formula X embodiment, R₁ and Z are both methyl and M and W are both absent.

In one Formula X embodiment, R₂ is selected from t-butyl, isopropyl, ethyl, methyl.

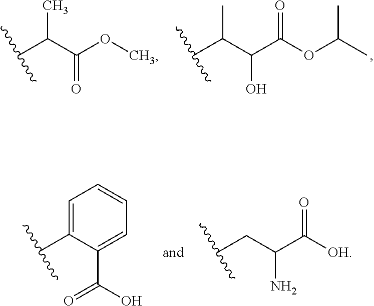

In a further Formula X embodiment, n is 1. In a further Formula X embodiment, R_{AA} is a proteinogenic amino acid side chain. In a further Formula X embodiment, R₁ and Z are both methyl and M and W are both absent.

R₂ is

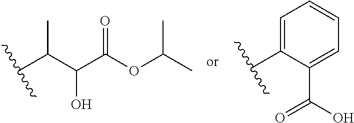

in another Formula X embodiment. In a further Formula X embodiment, R₁ and Z are both methyl and M and W are both absent.

In one Formula X embodiment, n is 1, 2 or 3 and at least one occurrence of R_{AA} is a proteinogenic amino acid side chain. In a further Formula X embodiment, R₁ and Z are both methyl and M and W are both absent.

In one Formula X embodiment, the carbamate or thiocarbamate prodrug of the present invention is a lactone of Formula X. In a further Formula X embodiment, R₁ and Z are both methyl and M and W are both absent.

n is 1, R₃ is cysteine and R_{AA} is a cysteine side chain in one Formula X embodiment. In a further Formula X embodiment, R₂ is H, methyl, isopropyl,

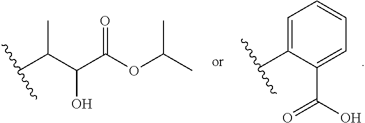

In a further Formula X embodiment, R₁ and Z are both methyl and M and W are both absent.

In some Formula X embodiments, n is 1, 2, 3, 4 or 5. In a further Formula X embodiment, R₁ and Z are both methyl and M and W are both absent.

In a preferred Formula X embodiment, the prodrug moiety of the compound of Formula X has one, two or three amino acids (i.e., n=1, 2 or 3), while R₂ and R₁ are both H.

In another Formula X embodiment, n is 2. At least one occurrence of R_{AA} is a proteinogenic amino acid side chain in a further Formula X embodiment. In a further Formula X embodiment, R₁ and Z are both methyl and M and W are both absent.

In yet another Formula X embodiment, R_{AA} is

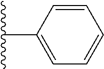

and n is 1. In a further Formula X embodiment, R₂ is H and R₃ is absent. In a further Formula X embodiment, R₁ and Z are both methyl and M and W are both absent.

In yet another Formula X embodiment, R_{AA} is

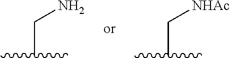

and n is 1. In a further Formula X embodiment, R₂ is H and R₃ is absent. In a further Formula X embodiment, R₁ and Z are both methyl and M and W are both absent.

In yet another Formula X embodiment, R_{AA} is

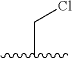

and n is 1. In a further Formula II embodiment, R₂ is H and R₃ is absent.

In yet another Formula X embodiment, R_{AA} is

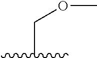

and n is 1. In a further Formula II embodiment, R₂ is H and R₃ is absent.

The present invention is also directed to a pharmaceutical composition comprising one or more of the meptazinol prodrugs of Formula X, and one or more pharmaceutically acceptable excipients.

In one embodiment, the meptazinol prodrugs of the present invention are directed to compounds of Formula XI:

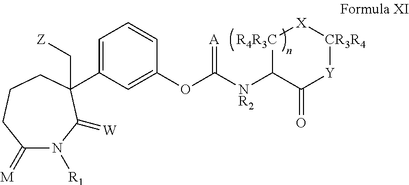

or a pharmaceutically acceptable salt thereof, wherein,

A is selected from O and S,

M and W are independently O or absent, and only one of M and W can be present on any one molecule,

Z is methyl, CH₂OH or COOH,

R₁ is H or methyl,

if Z is CH₂OH or COOH, M and W are both absent and R₁ is methyl,

if M or W is present, Z and R₁ are both methyl,

if R₁ is H, M and W are both absent while Z is methyl,

R₂ is H, alkyl or substituted alkyl,

R₃ and R₄ are independently selected from H, aryl, alkyl and substituted alkyl, and

n is an integer from 1 to 4.

In on Formula XI embodiment, M is O. In on Formula XI embodiment, W is O.

In one Formula XI embodiment, R₁ is H.

In one Formula XI embodiment, n is 1, X is S and A is O. Y is O in a further Formula XI embodiment. At least one occurrence of both R₃ and R₄ are methyl in a further embodiment.

In one Formula XI embodiment, n is 1, X is O and A is O. Y is O in a further Formula XI embodiment. At least one occurrence of both R₃ and R₄ are methyl in a further embodiment.

In one Formula XI embodiment, n is 2, X is S and A is O. Y is O in a further Formula XI embodiment. At least one occurrence of both R₃ and R₄ are methyl in a further embodiment.

In one Formula XI embodiment, n is 2, X is O and A is O. Y is O in a further Formula XI embodiment. At least one occurrence of both R₃ and R₄ are methyl in a further embodiment.

In one Formula XI embodiment, R₃ and R₄ between the X and Y atoms are both methyl. In a further Formula XI embodiment, n is 1. In still a further Formula XI embodiment, X is O and the additional R₂ group is methyl, while R₃ is H.

In one Formula XI embodiment, R₃ and R₄ between the X and Y atoms are both methyl. In a further Formula XI embodiment, n is 1. In still a further Formula XI embodiment, X is S and the additional R₃ group is methyl, while R₄ is H.

The present invention is also directed to a pharmaceutical composition comprising one or more of the meptazinol prodrugs of Formula XI, and one or more pharmaceutically acceptable excipients.

In one embodiment, the meptazinol prodrugs of the present invention are directed to compounds of Formula XII:

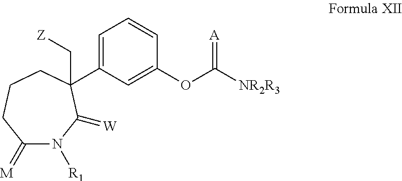

or a pharmaceutically acceptable salt thereof, wherein,

A is selected from O and S,

M and W are independently 0 or absent, and only one of M and W can be present on any one molecule,

Z is methyl, CH₂OH or COOH,

R₁ is H or methyl,

if Z is CH₂OH or COOH, M and W are both absent and R₁ is methyl,

if M or W is present, Z and R₁ are both methyl,

if R₁ is H, M and W are both absent while Z is methyl,

R₂ and R₃ are independently selected from H, aryl, alkyl and substituted alkyl group.

In one Formula XII embodiment, R₂ and R₃ are selected from propyl and butyl. In a further Formula XII embodiment, R₂ and R₃ are both propyl.

In one Formula XII embodiment, R₂ and R₃ are selected from hydrogen, methyl, propyl and butyl. In a further Formula XII embodiment, R₂ is hydrogen and R₃ is propyl.

In one Formula XII embodiment, R₂ and R₃ are selected from hydrogen, methyl, propyl and butyl. In a further Formula XII embodiment, R₂ is hydrogen and R₃ is butyl.

The present invention is also directed to a pharmaceutical composition comprising one or more of the meptazinol prodrugs of Formula XII, and one or more pharmaceutically acceptable excipients.

In one embodiment, the meptazinol prodrugs of the present invention are directed to compounds of Formula XIII:

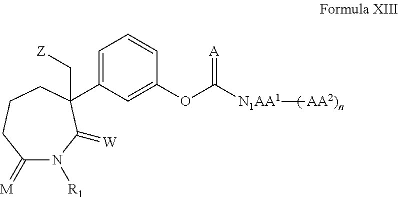

or a pharmaceutically acceptable salt thereof, wherein,

A is selected from O and S,

M and W are independently O or absent, and only one of M and W can be present on any one molecule,

Z is methyl, CH₂OH or COOH,

R₁ is H or methyl,

if Z is CH₂OH or COOH, M and W are both absent and R₁ is methyl,

if M or W is present, Z and R₁ are both methyl,

if R₁ is H, M and W are both absent while Z is methyl,

AA¹ is a proteinogenic amino acid, a β-amino acid (e.g., β-alanine) or pyroglutamic acid,

AA² is an α- or β-amino acid (e.g., valine),

n is an integer from 0 to 9;

N₁ is a nitrogen atom present in the first AA, and

In one Formula XIII embodiment, N₁ is the nitrogen atom of β-alanine

In one Formula XIII embodiment, n is 0 and AA¹ is pyroglutamic acid (pyroglutamate).

In one Formula XIII embodiment, N₁ is the nitrogen atom in a lysine side chain. In a further Formula XIII embodiment, n is 1 and the N-terminus of the lysine is bonded to valine (i.e., compound 27, described herein).

The present invention is also directed to a pharmaceutical composition comprising one or more of the meptazinol prodrugs of Formula XIII, and one or more pharmaceutically acceptable excipients.

In one embodiment, the meptazinol prodrugs of the present invention are directed to compounds of Formula XIII(A):

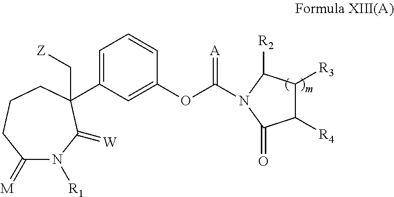

or a pharmaceutically acceptable salt thereof, wherein,

A is selected from O and S,

M and W are independently O or absent, and only one of M and W can be present on any one molecule,

Z is methyl, CH₂OH or COOH,

R₁ is H or methyl,

if Z is CH₂OH or COOH, M and W are both absent and R₁ is methyl,

if M or W is present, Z and R₁ are both methyl,

if R₁ is H, M and W are both absent while Z is methyl,

R₂, R₃ and R₄ are independently selected from hydrogen, aryl, alkyl, substituted alkyl group and carboxyl, and at least one occurrence of R₂, R₃ and R₄ is carboxyl, and

m is an integer from 1 to 3.

In one Formula XIII(A) embodiment, at least one carboxyl moiety of R₂, R₃ or R₄ is bound to an amino acid or peptide. In a further Formula XIII(A) embodiment, the amino acid bound to the at least one carboxyl moiety is valine. In still a further embodiment, R₂, R₃ and R₄ include only one carboxyl group.

In one Formula XIII(A) embodiment, m is 1. In a further Formula XIII(A) embodiment, A is O. In a further Formula XIII(A) embodiment, R₂ is carboxyl and R₃ and R₄ are both hydrogen.

In one Formula XIII(A) embodiment, m is 1. In a further Formula XIII(A) embodiment, A is S. In a further Formula XIII(A) embodiment, R₂ is carboxyl and R₃ and R₄ are both hydrogen.

In one Formula XIII(A) embodiment, m is 2. In a further Formula XIII(A) embodiment, A is O. In a further Formula XIII(A) embodiment, R₂ is carboxyl and R₃ and R₄ are both hydrogen.

In one Formula XIII(A) embodiment, m is 2. In a further Formula XIII(A) embodiment, A is S. In a further Formula XIII(A) embodiment, R₂ is carboxyl and R₃ and R₄ are both hydrogen.

In one Formula XIII(A) embodiment, m is 3. In a further Formula XIII(A) embodiment, A is O. In a further Formula XIII(A) embodiment, R₂ is carboxyl and R₃ and R₄ are both hydrogen.

In one Formula XIII(A) embodiment, m is 3. In a further Formula XIII(A) embodiment, A is S. In a further Formula XIII(A) embodiment, R₂ is carboxyl and R₃ and R₄ are both hydrogen.

In one Formula XIII(A) embodiment, at least one carboxyl moiety of R₂, R₃ or R₄ is bound to an amino acid or peptide. In a further Formula XIII(A) embodiment, the amino acid bound to the at least one carboxyl moiety is valine.

The present invention is also directed to a pharmaceutical composition comprising one or more of the meptazinol prodrugs of Formula XIII(A), and one or more pharmaceutically acceptable excipients.

In one embodiment, the carbamate and thiocarbamate prodrugs of the present invention are directed to compounds of Formula XIV:

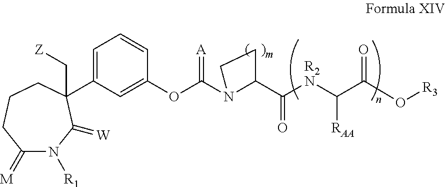

or a pharmaceutically acceptable salt thereof, wherein,

A is selected from O and S,

M and W are independently 0 or absent, and only one of M and W can be present on any one molecule,

Z is methyl, CH₂OH or COOH,

R₁ is H or methyl,

if Z is CH₂OH or COOH, M and W are both absent and R₁ is methyl,

if M or W is present, Z and R₁ are both methyl,

if R₁ is H, M and W are both absent while Z is methyl,

each occurrence of R₂ is independently hydrogen, alkyl or substituted alkyl,

m is an integer from 1 to 4 and n is an integer from 0 to 9,

R₃ is selected from hydrogen, C₁-C₄ alkyl, an amino acid (e.g., serine (—CH₂CH(NH₂)COOH)), or a substituted phenyl group (e.g., substituted with a carboxyl group, such as 2-COOH-phenyl) and an opioid,

If R₃ is an opioid, —O— is a hydroxylic oxygen present in the unbound opioid, and

each occurrence of R_{AA} is independently a proteinogenic or non-proteinogenic amino acid side chain.

In one Formula XIV embodiment, m is 1, n is 0 and R₃ is H.

In one Formula XIV embodiment, R₂ is not hydrogen.

In another Formula XIV embodiment, A is O, m is 2 n is 0, and R₂ and R₃ is hydrogen. In this embodiment, the prodrug moiety is proline carbamate.

In another Formula XIV embodiment, m is 1 and A is O. In a further Formula XIV embodiment, n is 0, 1 or 2. In a further Formula XIV embodiment, at least one R_{AA} is a proteinogenic amino acid side chain.

In yet another Formula XIV embodiment, m is 1 and A is S. In a further Formula XIV embodiment, n is 0, 1 or 2. In a further Formula XIV embodiment, at least one R_{AA} is a proteinogenic amino acid side chain.

In one Formula XIV embodiment, m is 2 and A is O. In a further Formula XIV embodiment, n is 0, 1 or 2. In a further Formula XIV embodiment, at least one R_{AA} is a proteinogenic amino acid side chain.

In one Formula XIV embodiment, m is 2 and A is S. In a further Formula XIV embodiment, n is 0, 1 or 2. In a further Formula XIV embodiment, at least one R_{AA} is a proteinogenic amino acid side chain.

In one Formula XIV embodiment, m is 3 and A is O. In a further Formula XIV embodiment, n is 0, 1 or 2. In a further Formula XIV embodiment, at least one R_{AA} is a proteinogenic amino acid side chain.

In another Formula XIV embodiment, m is 3 and A is S. In a further Formula XIV embodiment, n is 0, 1 or 2. In a further Formula XIV embodiment, at least one R_{AA} is a proteinogenic amino acid side chain.

In yet another Formula XIV embodiment, m is 4 and A is O. In a further Formula XIV embodiment, n is 0, 1 or 2. In a further Formula XIV embodiment, at least one R_{AA} is a proteinogenic amino acid side chain.

In another Formula XIV embodiment, m is 4 and A is S. In a further Formula XIV embodiment, n is 0, 1 or 2. In a further Formula XIV embodiment, at least one R_{AA} is a proteinogenic amino acid side chain.

In one Formula XIV embodiment, the opioid is meptazinol, R₃ is an opioid, and n is 1. In a further embodiment, R_{AA} is a valine side chain and R₃ is meptazinol.

In one Formula XIV embodiment, R₃ is selected from t-butyl, isopropyl, ethyl, methyl,

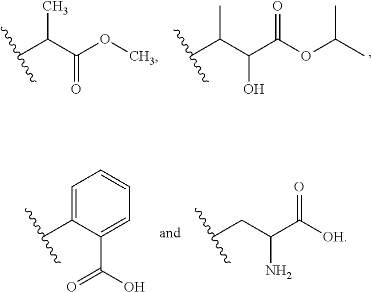

In a further Formula XIV embodiment, n is 1. In a further Formula XIV embodiment, R_{AA} is a proteinogenic amino acid side chain.

R₃ is

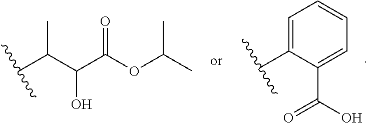

in another Formula XIV embodiment.

In one embodiment, the prodrug is a lactone of Formula XIV.

n is 1 in one Formula XIV embodiment. In a further Formula XIV embodiment, R₃ is H, methyl, isopropyl,

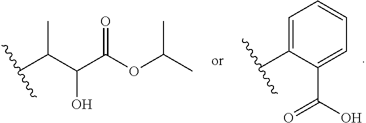

In a preferred Formula XIV embodiment, the prodrug moiety of the compound of Formula XIV has one, two or three amino acids, while R₃ is H.

In another Formula XIV embodiment, n is 2. At least one occurrence of R_{AA} is a proteinogenic amino acid side chain in a further Formula XIV embodiment.

In yet another Formula XIV embodiment, R_{AA} is

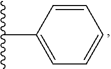

m is 1 or 2 and n is 1. In a further Formula XIV embodiment, and R₂ and R₃ are both H.

In yet another Formula XIV embodiment, R_{AA} is

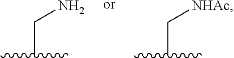

m is 1 or 2 and n is 1. In a further Formula XIV embodiment, R₂ and R₃ are both H.

In yet another Formula XIV embodiment, R_{AA} is

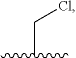

m is 1 or 2 and n is 1. In a further Formula XIV embodiment, R₂ and R₃ are both H.

In yet another Formula XIV embodiment, R_{AA} is

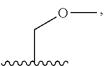

m is 1 or 2 and n is 1. In a further Formula XIV embodiment, R₂ and R₃ are both H.

In a preferred Formula XIV embodiment, the prodrug moiety of the compound of Formula XIV has one, two or three amino acids (i.e., n=1, 2 or 3), while R₂ is H.

In another Formula XIV embodiment, n is 2.

In yet another Formula XIV embodiment, n is 1 or 2 and each occurrence of R_{AA} is independently a proteinogenic amino acid side chain.

In yet another Formula XIV embodiment, n is 1 or 2 and at least one occurrence of R_{AA} is a non-proteinogenic amino acid side chain.

The present invention is also directed to a pharmaceutical composition comprising one or more of the meptazinol prodrugs of Formula XIV, and one or more pharmaceutically acceptable excipients.

In yet another embodiment, the carbamate and thiocarbamate prodrugs of the present invention are directed to compounds of Formula XV:

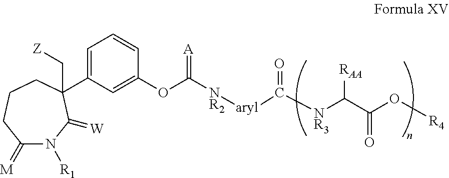

or a pharmaceutically acceptable salt thereof, wherein,

A is selected from O and S,

M and W are independently O or absent, and only one of M and W can be present on any one molecule,

Z is methyl, CH₂OH or COOH,

R₁ is H or methyl,

if Z is CH₂OH or COOH, M and W are both absent and R₁ is methyl,

if M or W is present, Z and R₁ are both methyl,

if R₁ is H, M and W are both absent while Z is methyl,

R₂ is independently selected from hydrogen, alkyl, substituted alkyl, cycloalkyl, and substituted cycloalkyl group,

Each occurrence of R₃ is independently selected from hydrogen, alkyl, substituted alkyl, an opioid, cycloalkyl, and substituted cycloalkyl group,

R₄ is independently selected from hydrogen, alkyl, substituted alkyl, cycloalkyl, substituted cycloalkyl group an an opioid,

If R₄ is an opioid, —O— is a hydroxylic oxygen present in the unbound opioid,

X is a nitrogen containing aryl group, where the nitrogen of the aryl group is bonded to the

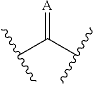

moiety (e.g., para-aminobenzoic acid),

Each occurrence of R_{AA} is independently a proteinogenic or non-proteinogenic amino acid side chain (e.g., R_{AA} can be isopropyl), and

n is an integer from 1 to 9.

In one Formula XV embodiment, R₄ is an opioid. In a further Formula XV embodiment, R₄ is meptazinol.

In one Formula XV embodiment, the

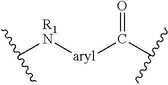

moiety is selected from

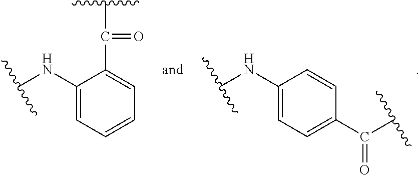

In a preferred Formula XV embodiment, the

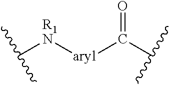

moiety is selected from

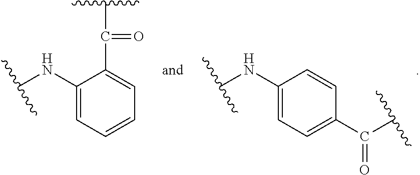

In one Formula XV embodiment, the opioid is meptazinol, R₄ is an opioid, and n is 1. In a further embodiment, R_{AA} is a valine side chain, R₄ is meptazinol and the

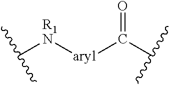

moiety is selected from

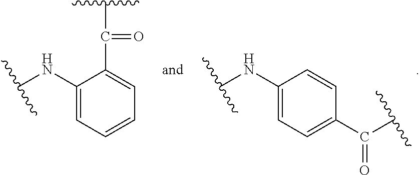

In one Formula XV embodiment, the

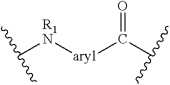

moiety is selected from

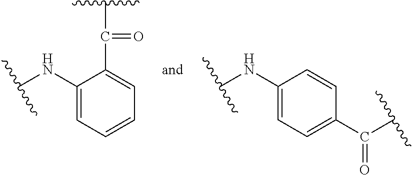

and R₄ is selected from t-butyl, isopropyl, ethyl, methyl,

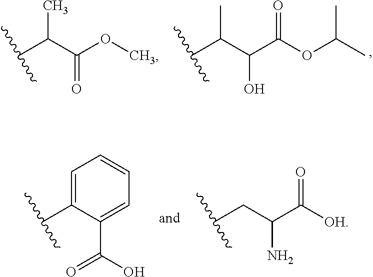

In a further Formula XV embodiment, n is 1. In a further Formula XV embodiment, R_{AA} is a proteinogenic amino acid side chain.

R₃ is

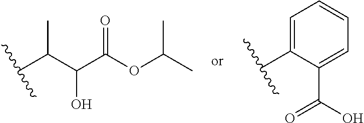

in another Formula XV embodiment.

n is 1, 2 or 3 and at least one occurrence of R_{AA} is a proteinogenic amino acid side chain in another Formula XV embodiment. In a further Formula XV embodiment, the

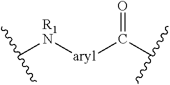

moiety is selected from

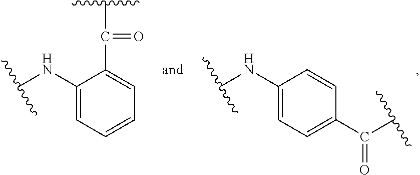

In one Formula XV embodiment, the prodrug is a lactone of Formula XV.

n is 1 in one Formula XV embodiment. In a further Formula XV embodiment, the

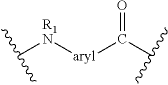

moiety is selected from

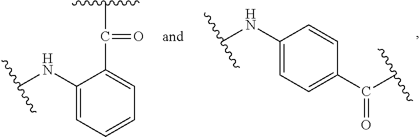

R₂ is H, methyl, isopropyl,

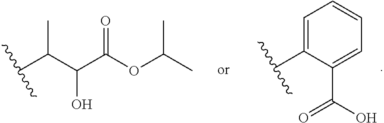

In a preferred Formula XV embodiment, the prodrug moiety of the compound of Formula XV has one, two or three amino acids, while R₂ is H.

In another Formula XV embodiment, n is 2 and the

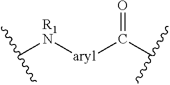

moiety is selected from

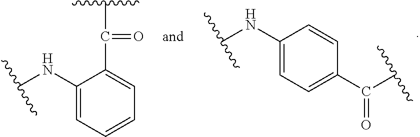

At least one occurrence of R_{AA} is a proteinogenic amino acid side chain in a further Formula XV embodiment.

In yet another Formula XV embodiment, the

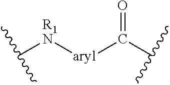

moiety is selected from

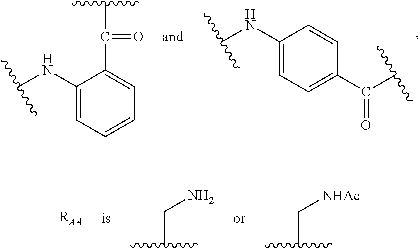

and n is 1. In a further Formula XV embodiment, R₂ and R₃ are both H. In a further Formula XV embodiment, the proteinogenic amino acid is selected from valine, isoleucine, alanine and leucine.

In yet another Formula XV embodiment, the

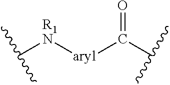

moiety is selected from

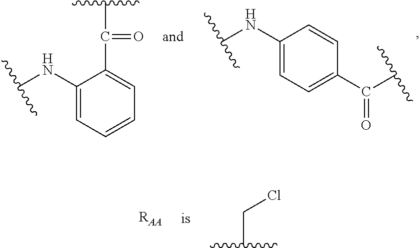

and n is 1. In a further Formula XV embodiment, R₁ and R₂ are both H. In a further Formula XV embodiment, the proteinogenic amino acid is selected from valine, isoleucine, alanine and leucine.

In yet another Formula XIV embodiment, the

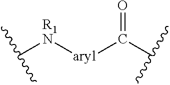

moiety is selected from

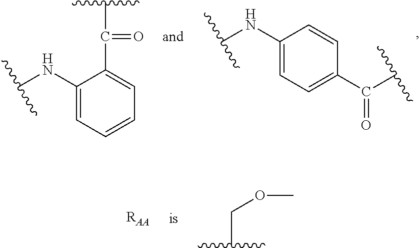

and n is 1. In a further Formula XV embodiment, R₁ and R₂ are both H. In a further Formula XV embodiment, the proteinogenic amino acid is selected from valine, isoleucine, alanine and leucine.

In a preferred Formula XV embodiment, the prodrug moiety of the compound of Formula XV has one, two or three amino acids (i.e., n=1, 2 or 3), while R₂ is H. In a further Formula XV embodiment, the proteinogenic amino acid is selected from valine, isoleucine, alanine and leucine.

In another Formula XV embodiment, n is 2. In a further Formula XV embodiment, the proteinogenic amino acid is selected from valine, isoleucine, alanine and leucine.

In yet another Formula XV embodiment, n is 1 or 2 and each occurrence of R_{AA} is independently a proteinogenic amino acid side chain. In a further Formula XV embodiment, the proteinogenic amino acid is selected from valine, isoleucine, alanine and leucine.

In yet another Formula XV embodiment, n is 1 or 2 and at least one occurrence of R_{AA} is a non-proteinogenic amino acid side chain.

The present invention is also directed to a pharmaceutical composition comprising one or more of the meptazinol prodrugs of Formula XV, and one or more pharmaceutically acceptable excipients.

Preferred amino acids described throughout the specification are all in the L configuration, however, the present invention also contemplates prodrugs of Formulae I-XV comprised of amino acids in the D configuration, or mixtures of amino acids in the D and L configurations.

In one embodiment, the present invention is directed to prodrug moiety permutations drawn from valine, leucine, isoleucine, alanine and glycine. These prodrug moieties can be used with any of the opioid analgesics described herein, including, but not limited to hydromorphone, oxymorphone, buprenorphine and meptazinol. Yet further embodiments may include permutations drawn from these nonpolar aliphatic amino acids with the nonpolar aromatic amino acids, tryptophan and tyrosine.

Alternatively, non-proteinogenic amino acid may also be used as the prodrug moiety or a portion thereof. If a non-proteinogenic amino acid is used in a peptide, the peptide can include only non-proteinogenic amino acids, or a combination of proteinogenic and non-proteinogenic amino acids.

Although Formulae IX-XV have been drawn with meptazinol as the opioid, it is to be understood that any opioid with a hydroxylic, carboxylic or hydroxylic function can be readily substituted for meptazinol to form a prodrug with the prodrug moieties of Formulae IX-XV. One of ordinary skill in the art will readily know how to make such a substitution.

Accordingly, in one embodiment, the carbamate and thiocarbamate prodrug moieties described above in Formulae IX-XV are used with at least one of the following opioid analgesics, to form an opioid prodrug conjugate—butorphanol, codeine, dezocine, dihydrocodeine, hydrocodone, hydroxymorphone, levorphanol, morphine, nalbuphine, oxycodone, and pentazocine.

Advantages of the Compounds of the Invention

Without wishing to be bound to any particular theory, it is believed that the amino acid or peptide portion of the opioid prodrug of the present invention selectively exploits the inherent di- and tripeptide transporter Pept1 within the digestive tract to effect absorption. It is believed that the opioid is subsequently released from the amino acid or peptide prodrug into the systemic circulation by hepatic and extrahepatic hydrolases that are, in part, present in plasma.

Furthermore, the prodrugs of the present invention temporarily inactivate the respective opioid, precluding any potential for local opioid action within the gut lumen on opioid or other receptors, thus, avoiding the adverse GI side effects such as constipation, commonly associated with opioid or other administration. Once absorbed, however, the opioid prodrug of the present invention is metabolized by plasma and liver esterases to the pharmacologically active opioid species which can then elicit its centrally mediated analgesic effects.

Reduction of the adverse GI side-effects associated with opioid administration is an advantage of using a prodrug of the present invention. As stated above, oral administration of a temporarily inactivated opioid would, during the absorption process, preclude access of active drug species to the μ-opioid receptors within the gut wall. The role that these peripheral μ-opioid receptors play on gut transit has recently been demonstrated by co-administration of peripherally confined narcotic antagonists such as alvimopan, and naloxone. (Linn and Steinbrook (2007). Tech in Reg. Anaes. and Pain Management 11, 27-32). Co-administration of these active agents with normally constipating opioid analgesics such as oxycodone has shown a reduction in effects on gut transit, without adversely affecting systemically mediated analgesia. Thus, oral administration of a transiently inactivated opioid may similarly avoid such problems of locally mediated constipation, without the need for co-administration of a peripheral μ-opioid antagonist.

Another potential advantage of the use of such prodrugs is a reduced likelihood of intravenous or intranasal abuse. The propensity for intravenous (i.v.) abuse is minimized by the slower rate formation of the active principal from the prodrug and consequent attainmment of Cₘₐₓ after i.v. dosing compared to that after i.v. dosing of the drug itself. Therefore, i.v. administration of the prodrug would give a “euphoric rush” less than the opioid itself.

Intranasal abuse of these amino acid/peptide prodrugs may be reduced by their negligible absorption from the nasal mucosa. This is due to the profound differences in physicochemical properties between parent opioids and the highly water soluble amino/peptide prodrugs disclosed herein. Opioid amino acid/peptide conjugates are not to be absorbed by simple diffusion due to their high water solubility and also adverse LogP values. Instead they would rely upon active transporters, such as Pept1 to assist in absorption, which while present in the gut, are essentially absent in the nasal mucosa.

In some embodiments, the oral bioavailability of the opioid provided by the compound of Formulae I-XV is higher than the oral bioavailability of the opioid, when administered alone.

Uses of the Invention

A method for reducing or eliminating pain with one or more opioid prodrugs of the present invention is provided. The method comprises administering to a subject in need thereof (e.g., an effective amount of) a prodrug of the present invention, or a composition of the present invention. In one embodiment, the method comprises administering to a subject in need thereof a carbamate or thiocarbamate prodrug of any of Formulae I-XV, or a composition thereof.

The types of pain that can be treated includes neuropathic pain and nociceptive pain. Other specific types of pain which can be treated with the opioid prodrugs of the present invention include, but are not limited to, acute pain, chronic pain, post-operative pain, pain due to neuralgia (e.g., post herpetic neuralgia or trigeminal neuralgia, pain due to diabetic neuropathy, dental pain, pain associated with arthritis or osteoarthritis, and pain associated with cancer or its treatment.

In the methods of treating pain, the prodrugs encompassed by the present invention may be administered in conjunction with other therapies and/or in combination with other active agents (e.g., other analgesics). For example, the prodrugs encompassed by the present invention may be administered to a subject in combination with other active agents used in the management of pain. An active agent to be administered in combination with the prodrugs encompassed by the present invention may include, for example, a drug selected from the group consisting of non-steroidal anti-inflammatory drugs (e.g., ibuprofen), anti-emetic agents (e.g., ondansetron, domerperidone, hyoscine and metoclopramide), and unabsorbed or poorly bioavailable opioid antagonists to reduce the risk of drug abuse (e.g., naloxone). In such combination therapies, the prodrugs encompassed by the present invention may be administered prior to, concurrent with, or subsequent to the other therapy and/or active agent. The prodrug and other active agent(s) may also be incorporated into a single dosage form.

In one embodiment, the present invention is directed to a method for increasing the oral bioavailability of an opioid. The method comprises administering, to a subject in need thereof, an effective amount of opioid carbamate or thiocarbamate prodrug of the present invention (i.e., a compound of Formula I-XV), or a composition thereof.

Another embodiment of the invention is a method of minimizing one or more gastrointestinal side effects in a patient receiving an unbound opioid analgesic, where the gastroinstestinal side effects result from or are aggravated by the administration of the opioid analgesic. The method comprises (i) discontinuing administration of the opioid analgesic to the patient, and (ii) administering to the patient an effective amount of an opioid carbamate or thiocarbamate prodrug of the present invention. According to one preferred embodiment, the opioid carbamate or thiocarbamate prodrug includes the same opioid as the discontinued opioid analgesic. The term “unbound opioid analgesic” refers to an opioid analgesic which is not a carbamate or thiocarbamate prodrug. This method is particularly useful for reducing gastrointestinal side effect(s) resulting from or aggravated by administration of the unbound opioid analgesic for pain relief.

The present invention is directed to the use of new amino acid and peptide prodrugs of established opioid analgesic agents and methods for decreasing gastrointestinal side-effects with the prodrugs. These prodrugs can comprise carbamate linked single amino acids or short peptides, preferably from 1 to 5 amino acids in length, attached to a hydroxylic or hydroxylic functional group within the drug molecule. The prodrug moiety renders these compounds temporarily inactive as opioid binding agents.

Without being bound by any particular theory, it is believed that the subject receiving the prodrug will avoid, or experience reduced GI side effects (e.g., emesis, constipation) associated with opioid compounds that bind to the μ-opioid, cholinergic, or other receptors located in the gut. Once absorbed, however, such prodrugs would be metabolized by plasma and liver enzymes to the pharmacologically active opioid species which can then elicit its centrally mediated analgesic effects. However, it is to be understood that the present invention is not limited to the foregoing hypothesis, and the prodrug compounds and methods disclosed herein can act by some other mechanism to reduce or eliminate GI side effects associated with unmodified opioid analgesics.

Accordingly, the present invention provides compounds, compositions and methods for reducing the GI side effects associated with opioid analgesics that are mediated by the μ-opioid or cholinergic receptors resident in the gut.

Additionally, the invention provides compositions for, and methods of reducing gastrointestinal side effects brought on by classical opioid analgesics, as well as pain from POI.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. For highly potent agents such as buprenorphine, the daily dose requirement may, for example, range from 0.5 to 50 mg, preferably from 1 to 25 mg, and more preferably from 1 mg to 10 mg. For less potent agents such as meptazinol, the daily dose requirement may, for example, range from 1 mg to 1600 mg, preferably from 1 mg to 800 mg and more preferably from 1 mg to 400 mg.

The doses referred to throughout the specification refer to the amount of the opioid free base in the particular compound.

If oxymorphone is the opioid used in the present invention, doses can be derived from the commercially available oxymorphone products Opana®, Numorphan® and Numorphone® factoring in any differences in oral bioavailability.

Salts, Solvates, Stereoisomers, Derivatives of the Compounds Employed in the Present Invention

The methods of the present invention further encompass the use of salts, solvates, stereoisomers of the opioid prodrugs described herein, for example salts of the prodrugs of Formulae I-XV, given above.

Typically, a pharmaceutically acceptable salt of an opioid prodrug used in the practice of the present invention is prepared by reaction of the opioid prodrug with a desired acid or base as appropriate. The salt may precipitate from solution and be collected by filtration or may be recovered by evaporation of the solvent. For example, an aqueous solution of an acid such as hydrochloric acid may be added to an aqueous suspension of the opioid prodrug and the resulting mixture evaporated to dryness (lyophilized) to obtain the acid addition salt as a solid. Alternatively, the opioid prodrug may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

The acid addition salts of the opioid prodrugs may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metals or organic amines. Examples of metals used as cations are sodium, potassium, magnesium and calcium. Examples of suitable amines are N,N′-dibenzylethylenediamine, chloroprocaine, choline, diethanolamine, dicyclohexylamine, ethylenediamine, N-methylglucamine, and procaine.

The base addition salts of said acidic compounds are prepared by contacting the free acid form with a sufficient amount of the desired base to produce the salt in the conventional manner. The free acid form may be regenerated by contacting the salt form with an acid and isolating the free acid.

Compounds useful in the practice of the present invention may have both a basic and an acidic center and may therefore be in the form of zwitterions.

Those skilled in the art of organic chemistry will appreciate that many organic compounds can form complexes, i.e., solvates, with solvents in which they are reacted or from which they are precipitated or crystallized, e.g., hydrates with water. The salts of compounds useful in the present invention may form solvates such as hydrates useful therein. Techniques for the preparation of solvates are well known in the art (see, e.g., Brittain. Polymorphism in Pharmaceutical Solids. Marcel Decker, New York, 1999.). The compounds useful in the practice of the present invention can have one or more chiral centers and, depending on the nature of individual substituents, they can also have geometrical isomers.

Individual isomers of the opioid prodrugs described herein may be used to practice the present invention. The description or naming of a particular compound in the specification and claims is intended to include both individual enantiomers and mixtures, racemic or otherwise, thereof. Methods for the determination of stereochemistry and the resolution of stereoisomers are well-known in the art.

Pharmaceutical Compositions Comprising the Opioid Peptide Prodrug

While it is possible that, for use in the methods of the invention, the prodrug may be administered as the unadulterated substance, it is preferable to present the active ingredient in a pharmaceutical formulation, e.g., wherein the agent is in admixture with a pharmaceutically acceptable carrier selected with regard to the intended route of administration and standard pharmaceutical practice.

Therefore, in some embodiments, the present invention is directed to a composition comprising an opioid prodrug and a pharmaceutically acceptable excipient. The prodrug can be any prodrug described herein, including a prodrug of Formulae I-IX.

The formulations of the present invention can be administered from one to four times daily, depending on the dosage. The formulations of the invention may be immediate-release dosage forms, i.e. dosage forms that release the prodrug at the site of absorption immediately, or controlled-release dosage forms, i.e., dosage forms that release the prodrug over a predetermined period of time. Controlled release dosage forms may be of any conventional type, e.g., in the form of reservoir or matrix-type diffusion-controlled dosage forms; matrix, encapsulated or enteric-coated dissolution-controlled dosage forms; or osmotic dosage forms. Dosage forms of such types are disclosed, for example, in Remington, The Science and Practice of Pharmacy, 20th Edition, 2000, pp. 858-914. The formulations of the present invention can be administered from one to six times daily, depending on the dosage form and dosage.

Prodrugs of hydroxylic opioid analgesics which do not result in sustained plasma drugs levels due to continuous generation of the opioid analgesic from a plasma reservoir of prodrug may require formulations that provide a sustained release of the opioid analgesic. For example, formulations that offer gastroretentive or mucoretentive benefits, analogous to those used in metformin products such as Glumetz® or Gluphage XR®, may be employed. An example of the former is a drug delivery system known as Gelshield Diffusion™ Technology while an example of the latter is a so-called Acuform™ delivery system. In both cases, the concept is to retain drug in the stomach, slowing drug passage into the ileum, maximizing the period over which absorption take place and effectively prolonging plasma drug levels. Other drug delivery systems affording delayed progression along the GI tract may also be of value.

In one aspect, the present invention provides a pharmaceutical composition comprising at least one active pharmaceutical ingredient (i.e., an opioid-peptide prodrug), or a pharmaceutically acceptable derivative (e.g., a salt or solvate) thereof, and, optionally, a pharmaceutically acceptable carrier. In particular, the invention provides a pharmaceutical composition comprising a therapeutically effective amount of at least one opioid prodrug of the present invention, or a pharmaceutically acceptable derivative thereof, and, optionally, a pharmaceutically acceptable carrier.

For the methods of the invention, the prodrug employed may be used in combination with other therapies and/or active agents (e.g., other analgesics). Accordingly, the present invention provides, in a further aspect, a pharmaceutical composition comprising at least one compound useful in the practice of the present invention, or a pharmaceutically acceptable derivative thereof, a second active agent, and, optionally a pharmaceutically acceptable carrier.

For example, the prodrugs of the present invention may be administered to a subject in combination with other active agents used in the management of pain. An active agent to be administered in combination with the prodrugs encompassed by the present invention may include, for example, a drug selected from the group consisting of non-steroidal anti-inflammatory drugs (e.g., acetaminophen and ibuprofen), anti-emetic agents (e.g., ondansetron, domerperidone, hyoscine and metoclopramide), unabsorbed or poorly bioavailable opioid antagonists to reduce the risk of drug abuse (e.g., naloxone). In such combination therapies, the prodrugs encompassed by the present invention may be administered prior to, concurrent with, or subsequent to the other therapy and/or active agent.

When combined in the same formulation it will be appreciated that the two compounds must be stable and compatible with each other and the other components of the formulation. When formulated separately they may be provided in any convenient formulation, conveniently in such manner as are known for such compounds in the art.

The prodrugs used herein may be formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro, 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may be also used.

The compounds used in the invention may be milled using known milling procedures such as wet milling to obtain a particle size appropriate for tablet formation and for other formulation types. Finely divided (nanoparticulate) preparations of the compounds may be prepared by processes known in the art, for example see International Patent Application No. WO 02/00196 (SmithKline Beecham).

The compounds and pharmaceutical compositions of the present invention are intended to be administered orally (e.g., as a tablet, sachet, capsule, pastille, pill, boluse, powder, paste, granules, bullets or premix preparation, ovule, elixir, solution, suspension, dispersion, gel, syrup or as an ingestible solution). In addition, compounds may be present as a dry powder for constitution with water or other suitable vehicle before use, optionally with flavoring and coloring agents. Solid and liquid compositions may be prepared according to methods well-known in the art. Such compositions may also contain one or more pharmaceutically acceptable carriers and excipients which may be in solid or liquid form.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Examples of useful pharmaceutically acceptable coatings for the oral compositions, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulfate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetriacetic acid (EDTA), thiourea, tocopherol and butyl hydroxyanisole.

The pharmaceutical compositions of the invention may contain from 0.01 to 99% weight per volume of the active material.

The present invention is further illustrated by reference to the Examples below. However, it should be noted that these Examples, like the embodiments described above, are illustrative and are not to be construed as restricting the enabled scope of the invention in any way.

### EXAMPLES

Preparation of Prodrugs Employed in the Invention

Compounds employed in the present invention and derivatives thereof may be prepared by the general methods outlined hereinafter.

Chemicals were purchased primarily from Aldrich Chemical Company, Gillingham, Dorset and Alfa Aesar, Morecambe, Lancashire, U.K. and were used without further purification. Solvents utilized were anhydrous. Gasoline employed was the fraction boiling in the range 40-60° C.

TLC was carried out using aluminum plates pre-coated with silica gel (Kieselgel 60 F₂₅₄, 0.2 mm, Merck, Darmstadt, Germany). Visualization was by UV light or KMnO₄ dip. Silica gel (‘flash’, Kieselgel 60) was used for medium pressure chromatography.

¹H NMR spectra were recorded on a Bruker Avance BVT3200 spectrometer using deuterated solvents as internal standards.

Combustion analyses were performed by Advanced Chemical and Material Analysis, Newcastle University, U.K. using a Carlo-Erba 1108 elemental analyser.

### Example 1

### Generic Route of Synthesis of Amino Acid Carbamate Conjugates of Opioids

A route to hydroxylic opioid prodrugs as HCl or TFA salts via amino acid tent-butyl esters (with valine as an example) is given in Scheme 4, below. One of ordinary skill in the art would readily understood how to substitute a thiocarbonyl group for the carbonyl group in this scheme.

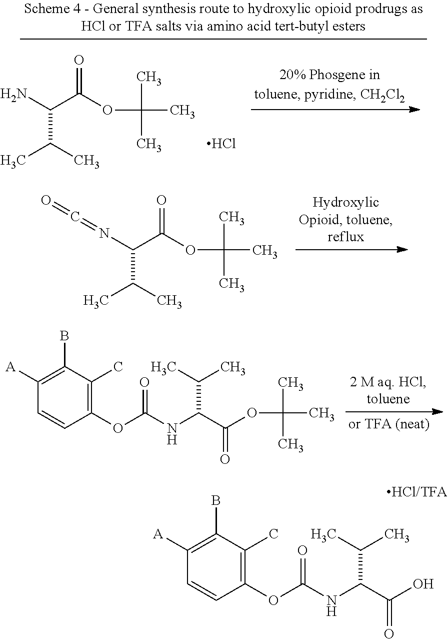

A route to hydroxylic opioid prodrugs via amino acid benzyl esters is given in Scheme 5, below (using valine as an example). One of ordinary skill in the art would readily understood how to substitute a thiocarbonyl group for the carbonyl group in this scheme.

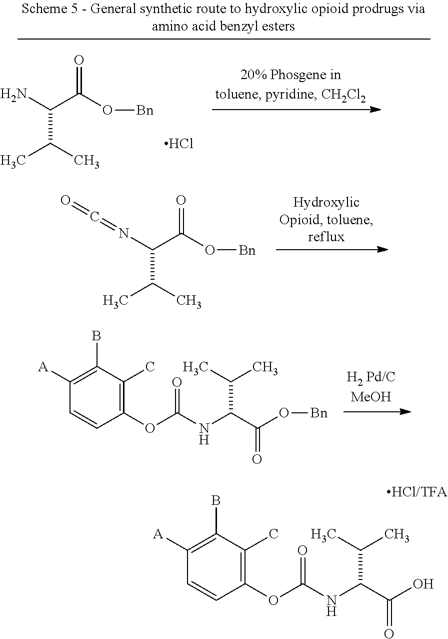

A general route to hydroxylic opioid prodrugs via a chloroformate intermediate is given in Scheme 6, below (using pyroglutamate and a generic opioid as an example). It is to be understood that any opioid with a hydroxylic function may be employed in this synthesis scheme. One of ordinary skill in the art would readily understood how to substitute a thiocarbonyl group for the carbonyl group in this scheme, in order to make a thiocarbamate bond.

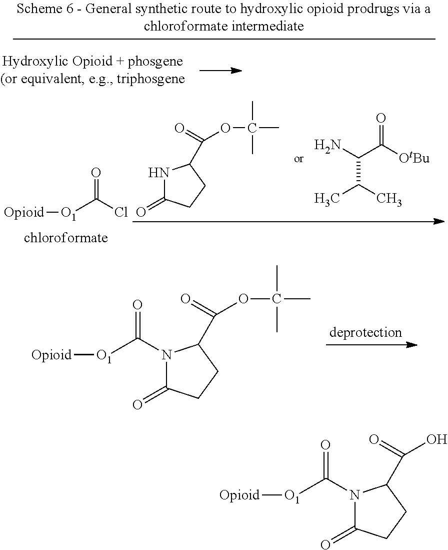

A general route to bis-acylated opioid-amino acid prodrug is given in Scheme 7, below (using valine and a generic opioid as an example). It is to be understood that any opioid with a hydroxylic function may be employed in this synthesis scheme. Further, any protected amino acid or protected peptide can be employed in this reaction scheme. One of ordinary skill in the art would readily understood how to substitute a thiocarbonyl group for the carbonyl group in this scheme, to make a thiocarbonate bond.

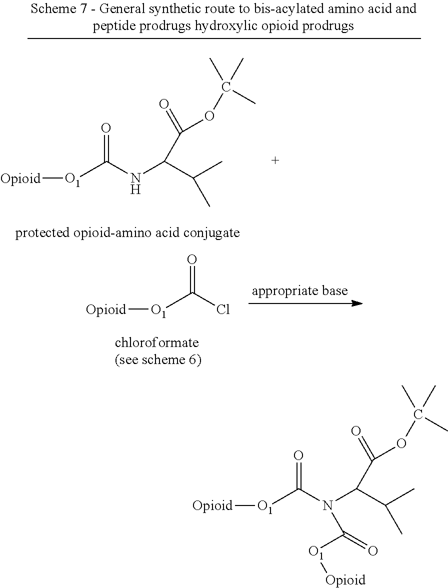

The first route (Scheme 4) is suitable for non-acid sensitive hydroxylic opioids, whereas the second route (Scheme 5) is suitable for those which are acid sensitive but do not contain any reducible functionalities such as double bonds.

The methods taught in U.S. patent application Ser. Nos. 12/356,028 and 12/356,034, as well as International Application Nos. PCT/U.S.09/31404 and PCT/U.S.09/31408, all are incorporated herein by reference in their entireties.

The following compounds, using meptazinol and valine as examples of a hydroxylic opioid and amino acid, respectively, can be made by these methods. It is to be understood that other opioids can be readily substituted for meptazinol, for conjugation to the various prodrug moieties described herein. One of ordinary skill in the art will also readily know how to substitute another amino acid or peptide, where desired.

| | Prodrug | Structure |
|---|---|---|
| 1 | MVC ter-Butyl ester | 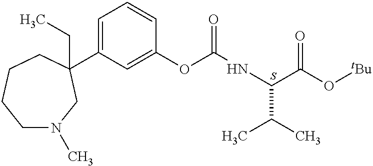 |
| 2 | MVC Isopropyl ester | 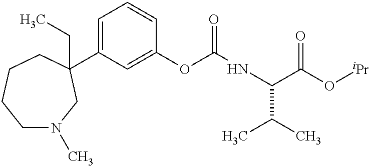 |
| 3 | MVC ethyl ester | 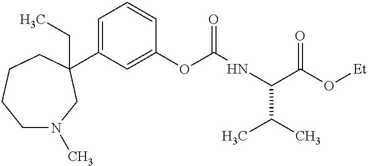 |
| 4 | MVC [isopropyl-(S)-lactate] ester | 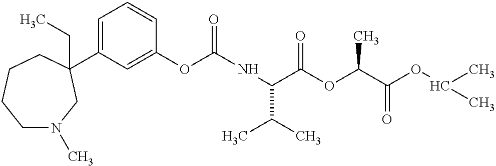 |
| 5 | MVC Salicyclic acid ester | 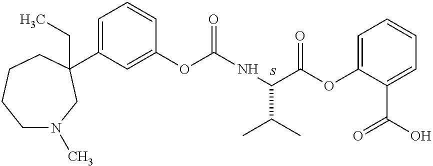 |
| 6 | MVC (S)serine ester | 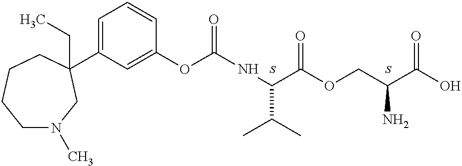 |
| 7 | Meptazinol homo-serine lactone carbamate | 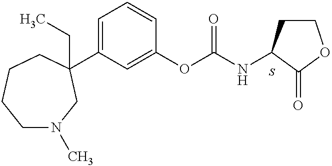 |
| 8 | Meptazinol aminomaalonic acid carbamate | 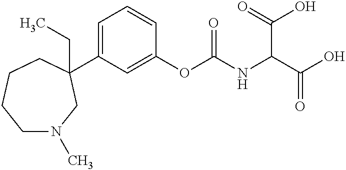 |
| 9 | Meptazinol cysteine carbamate | 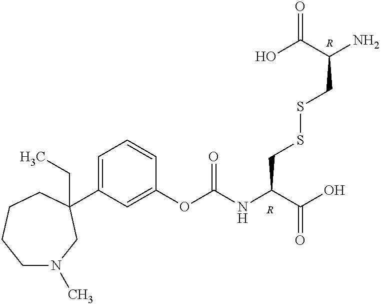 |
| 10 | Meptazinol β-alanine-valine carbamate | 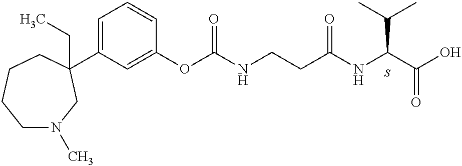 |
| 11 | Meptazinol mono-propyl carbamate | 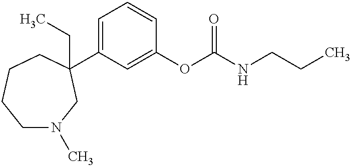 |
| 12 | Meptazinol di-propyl carbamate | 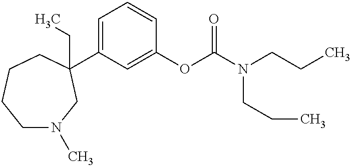 |
| 13 | Meptazinol sarcosine carbamate | 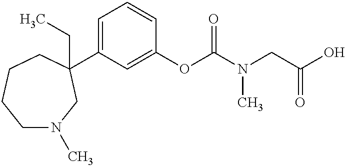 |
| 14 | Meptazinol (O-methyl serine) carbamate | 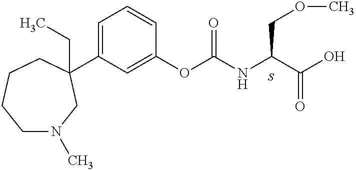 |
| 15 | Meptazinol β-(acetylamino)alanine carbamate | 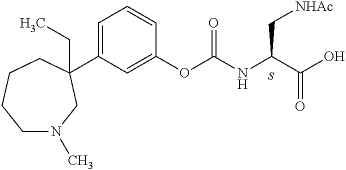 |
| 16 | Meptazinol β-aminoalanine carbamate | 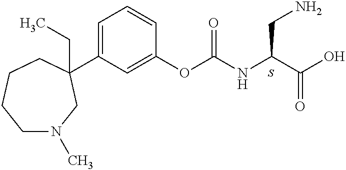 |
| 17 | Meptazinol (isopropylidene-threonine) carbamate | 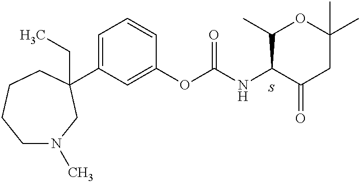 |
| 18 | Meptazinol phenylglycine carbamate | 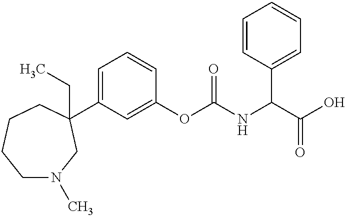 |
| 19 | Meptazinol proline carbamate | 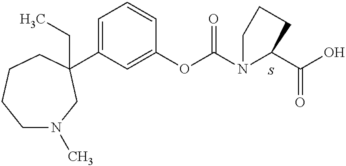 |
| 20 | Meptazinol (isopropylidene-cysteine) carbamate | 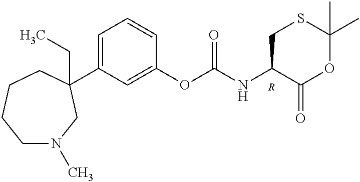 |
| 21 | Meptazinol (isopropylidene-homo-cysteine) carbamate | 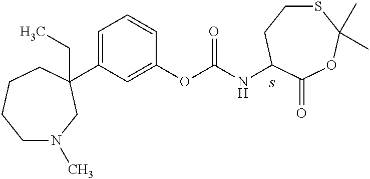 |
| 22 | Meptazinol β-chloroalanine carbamate | 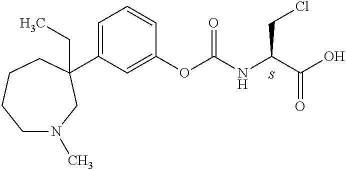 |
| 23 | Des-methyl meptazinol-S-valine carbamate | 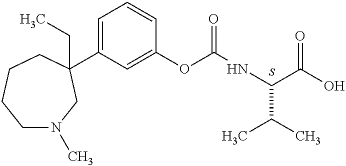 |
| 24 | 2-Oxomeptazinol-S-valine carbamate | 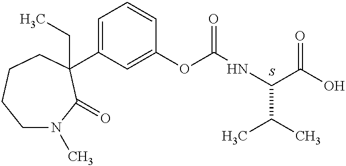 |
| 25 | 7-Oxomeptazinol-S-valine carbamate | 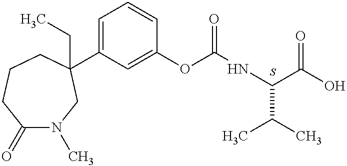 |
| 26 | Meptazinol valine thiocarbamate | 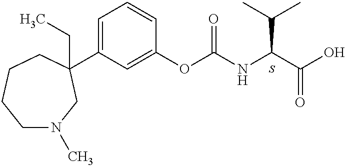 |
| 27 | Meptazinol valine-lysine side-chain carbamate H-Val-Lys(CO.OMeptazinol)-OH | 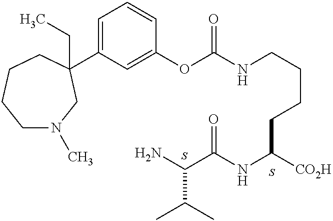 |
| 28 | Meptazinol pyrroglutamate carbamate | 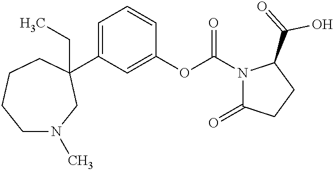 |
| 29 | Bis-Meptazinol valine carbamate | 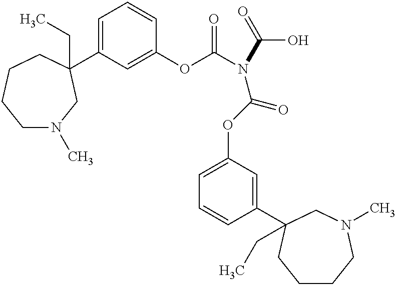 |
| 30 | Meptazinol para aminobenoic acid valine carbamate | 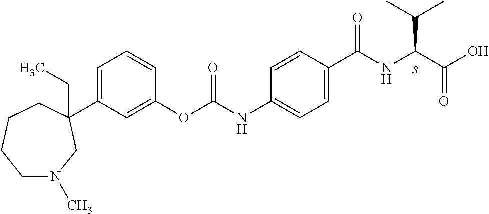 |

### Example 2

### Synthesis of Des-Methyl Meptazinol Hydrobromide

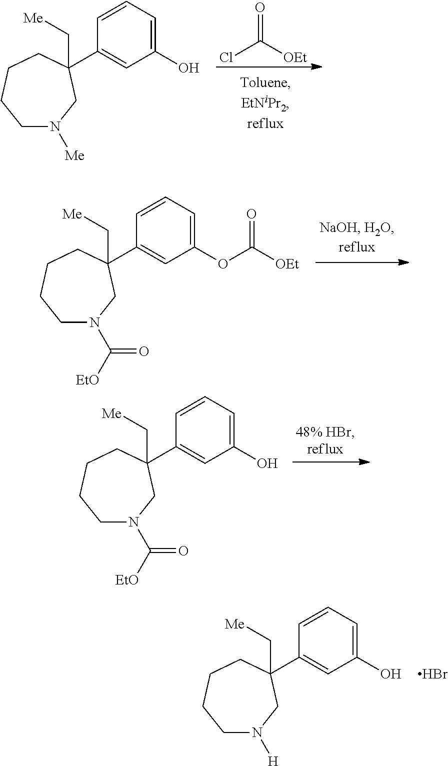

### Example 3

### Synthesis of Des-Methyl Meptazinol-S-Valine Carbamate Trifluoroacetate

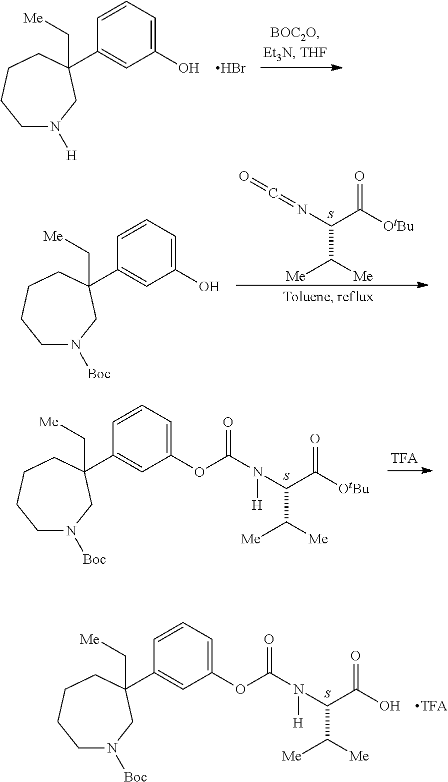

### Example 4

### Synthesis of 2-Oxomeptazinol-S-Valine Carbamate Trifluoroacetate

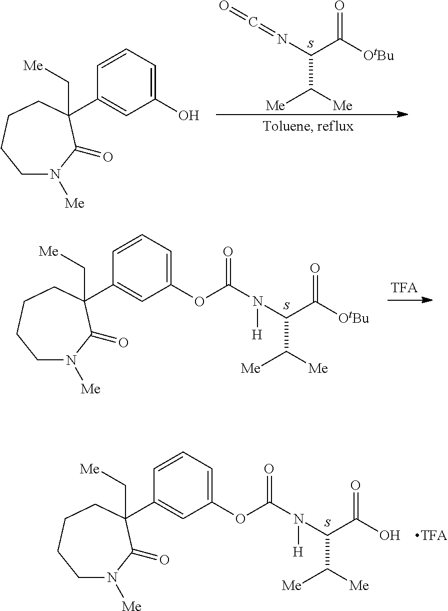

### Example 5

### Synthesis of 7-Oxomeptazinol Valine Carbamate Trifluoroacetate

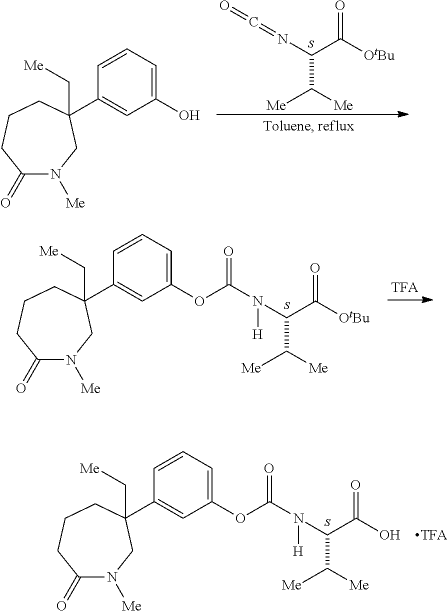

### Example 6

### Synthesis of Ethyl-Hydroxylated Meptazinol

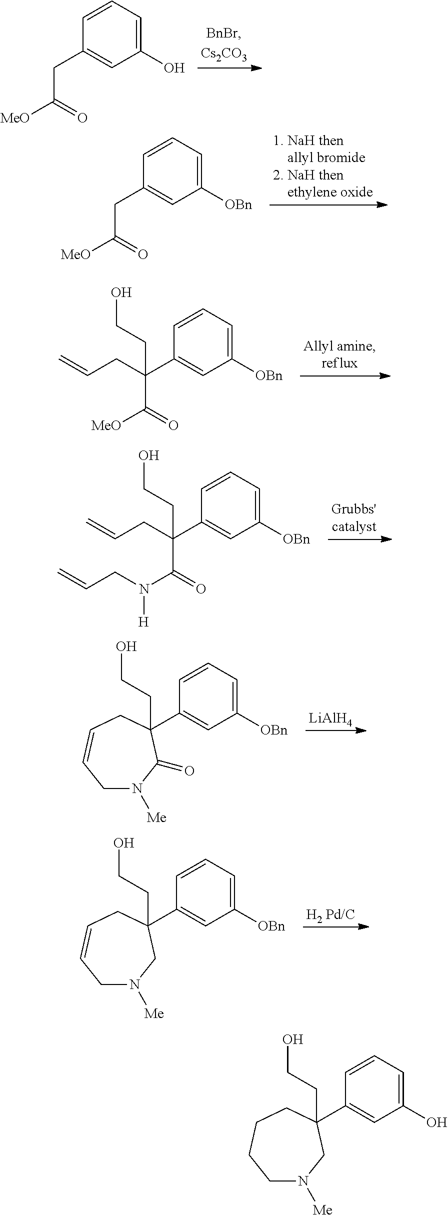

### Example 7

### Synthesis of Ethyl-Carboxylated Meptazinol

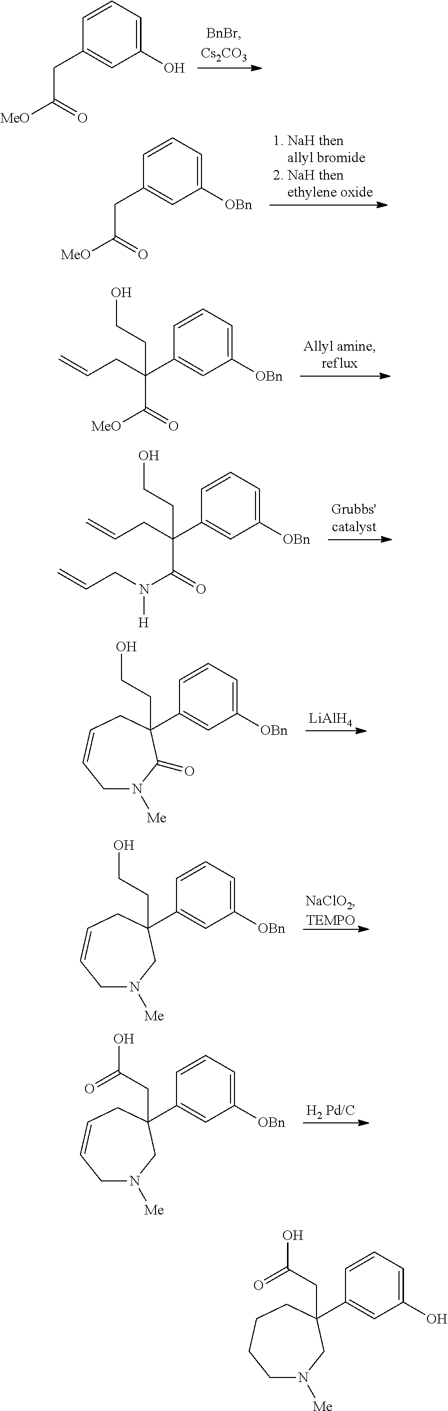

### Example 8

### Assessment of Cholinergic Effects of Meptazinol Carbamate and Thiocarbamate Prodrugs in Isolated Gut Preparation

The direct effects of meptazinol and the meptazinol carbamate and thiocarbamate prodrugs are assessed, using an ex vivo isolated gut smooth muscle model. Circular muscle strips of rat and human colon are dissected and set up in an organ bath system. Changes in smooth muscle force production are monitored using a pressure transducer. Nerves within the muscle strips are stimulated using an electrical field, which creates paced contractions of the smooth muscle. The potential influence of these compounds on gut motility is then assessed by measuring the size of contractions.

### Example 9

### Demonstration of In Vivo Bioavailability of Opioids from their Amino Acid Prodrugs in does or Minipigs

Test substances (i.e., opioid and selected prodrugs) are administered by oral gavage to a group of dogs or minipigs in a crossover design. The characteristics of the test animals are set out in Table 2, below.

TABLE 2
Characteristics of experimental animals for use in study

| | |
|---|---|
| Species | Dog (oxymorphone, buprenorphine, meptazinol) |
| | or Minipigs (hydromorphone) |
| Type | Beagle dogs or Gottingen minpigs |
| Number and sex | 5 males |
| Approximate age | 3-4 months at the start of treatment |
| Approx. bodyweight | 7-9 kg at the start of treatment |
| Source | Huntingdon Life Sciences stock |

Blood samples are taken at various times after administration and submitted to analysis for the parent drug and prodrug using a validated LC-MS-MS assay. Pharmacokinetic parameters derived from the plasma analytical data are determined using Win Nonlin.

### Example 10

### Assessment of Emesis Induced by Meptazinol and Meptazinol Carbamate and Thiocarbamate Prodrugs in the Ferret

Female ferrets, starved overnight, are pre-treated the following morning with naloxone by subcutaneous injection (0.5 mg/kg) using a dose volume of 1 mL/kg. This is administered to minimize the otherwise profound CNS depression seen at these relatively high doses of meptazinol. Approximately 15 minutes later the animals receive, by oral gavage, either an aqueous solution of meptazinol HCl or meptazinol prodrug using a constant dose volume of 5 mL/kg. The animals were continuously observed for 2 hours post oral treatment and any incidences of retching and vomiting are recorded.

### Example 11

### In Vitro Assessment of Stability of Various Opioid Amino Acid Carbamates Under Conditions Prevailing in Gut

Methodology

Inherent chemical and biological stability of the prodrug under the conditions prevailing in the GI tract is a mandatory requirement. If the opioid prodrug should be prematurely hydrolyzed it would negate the opportunity to deliver, systemically, the intact prodrug from which the active drug may be continuously generated. To investigate this various opioid amino acid valine carbamate and thiocarbamate prodrugs are incubated at 37° C. in simulated gastric and simulated intestinal juice (USP defined composition) for 2 hours. The remaining concentration of the prodrug is assayed by HPLC. For comparative purposes, stabilities in three other standard media are also determined.

Patents, patent applications, publications, product descriptions, and protocols which are cited throughout this application are incorporated herein by reference in their entireties.

The embodiments illustrated and discussed in this specification are intended only to teach those skilled in the art the best way known to the inventors to make and use the invention. Nothing in this specification should be considered as limiting the scope of the present invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. A compound of Formula I: 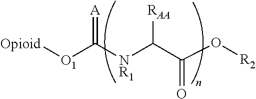
or a pharmaceutically acceptable salt thereof, wherein
O₁ is a hydroxylic oxygen present in an unbound opioid molecule,
A is O or S,
each occurrence of R₁ is independently hydrogen, alkyl or substituted alkyl,
R₂ is a C₁-C₄ alkyl, an amino acid, a substituted phenyl group, a substituted alkyl group, t-butyl, isopropyl, ethyl, methyl, 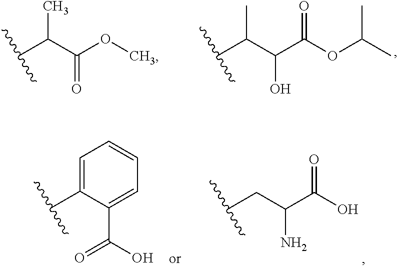
n is an integer from 1 to 9,
each occurrence of R_{AA} is independently a proteinogenic or a non-proteinogenic amino acid side chain, and
the opioid is selected from the group consisting of butorphanol, buprenorphine, codeine, dezocine, dihydrocodeine, hydromorphone, levorphanol, meptazinol, morphine, nalbuphine, oxycodone, oxymorphone, pentazocine, active metabolites thereof.

2. The compound of claim 1 wherein R₂ is serine or threonine.

3. The compound of claim 1 wherein A is O, R₁ is hydrogen, R₂ is serine or threonine, and n is 1.

4. The compound of claim 1 wherein R_{AA} is the amino acid side chain of valine.

5. The compound of claim 1 wherein the opioid is meptazinol, A is O, R₁ is hydrogen, R_{AA} is the side chain or valine, n is 1, and R₂ is serine.

6. A pharmaceutical composition comprising the compound of claim 5 and a pharmaceutically acceptable excipient.

7. The compound of claim 5 comprising a dihydrochloride salt represented by the formula:

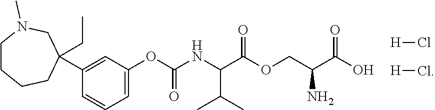

8. A compound represented by the formula:

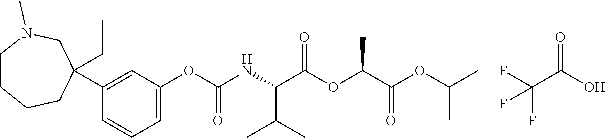

9. A pharmaceutical composition comprising the compound of claim 8 and a pharmaceutically acceptable excipient.

10. A method for reducing pain which comprises administering a compound of claim 1 to a patient suffering from pain.
